(19) United States
(12) Patent Application Publication
(10) Pub. No.: US 2010/0152468 A1
(43) Pub. Date: Jun. 17, 2010

(54) PROCESS FOR THE SYNTHESIS OF RAMELTEON AND ITS INTERMEDIATES

(75) Inventors: Vinod Kumar Kansal, Haryana (IN); Dhirenkumar N. Mistry, Gujarat (IN); Sanjay L. Vasoya, Gujarat (IN); Arpan M. Jadav, Gujarat (IN); Pratish Dadhaniya, Noida (IN); Jitendra Nalawade, Noida (IN)

Correspondence Address: KENYON & KENYON LLP, ONE BROADWAY, NEW YORK, NY, 10004, US

(73) Assignee: Teva Pharmaceutical Industries Ltd.

(21) Appl. No.: 12/580,679

(22) Filed: Oct. 16, 2009

Related U.S. Application Data
(60) Provisional application No. 61/106,070, filed on Oct. 16, 2008.
(60) Provisional application No. 61/196,858, filed on Oct. 20, 2008.
(60) Provisional application No. 61/111,973, filed on Nov. 6, 2008.

(51) Int. Cl. C07D 307/77

(52) U.S. Cl. 549/458

(57) ABSTRACT

The present invention provides processes and intermediates for the synthesis of ramelteon.

### CROSS REFERENCE

The present invention claims the benefit of the following United States Provisional Patent Application Nos. 61/106,070, filed Oct. 16, 2008; 61/196,858, filed Oct. 20, 2008; 61/111,973, filed Nov. 6, 2008. The contents of these applications are incorporated herein by reference.

### FIELD OF THE INVENTION

The present invention relates to novel synthesis of (S)—N-[2-(1,6,7,8-tetrahydro-2H-indeno-[5,4-b]furan-8-yl)ethyl] propionamide, i.e. ramelteon.

### BACKGROUND OF THE INVENTION

Ramelteon is a melatonin receptor agonist with both high affinity for melatonin MT1 and MT2 receptors and selectivity over the MT3 receptor. The empirical formula for ramelteon is C₁₆H₂₁NO₂, and its molecular weight is 259.34. Ramelteon is freely soluble in methanol, ethanol, DMSO, and 1-octanol, and slightly soluble in water and aqueous buffer. Ramelteon has the following chemical structure:

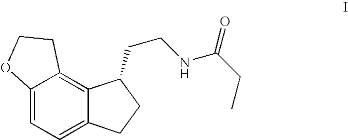

Ramelteon is the active ingredient in trademarked ROZEREM®, and is approved by the United States Food and Drug Administration for the treatment of insomnia characterized by difficulty with sleep onset.

Different processes for preparing (S)—N-[2-(1,6,7,8-tetrahydro-2H-indeno-[5,4-b]furan-8-yl)ethyl]propionamide, i.e. ramelteon, are disclosed in U.S. Pat. No. 6,034,239, JP 11080106, JP 11140073 and WO 2006/030739. Other processes are disclosed in WO2009/106966, WO2008/150933, WO2008/151170, and WO2009/56993.

U.S. Pat. No. 6,034,239 describes the following processes for the preparation of ramelteon:

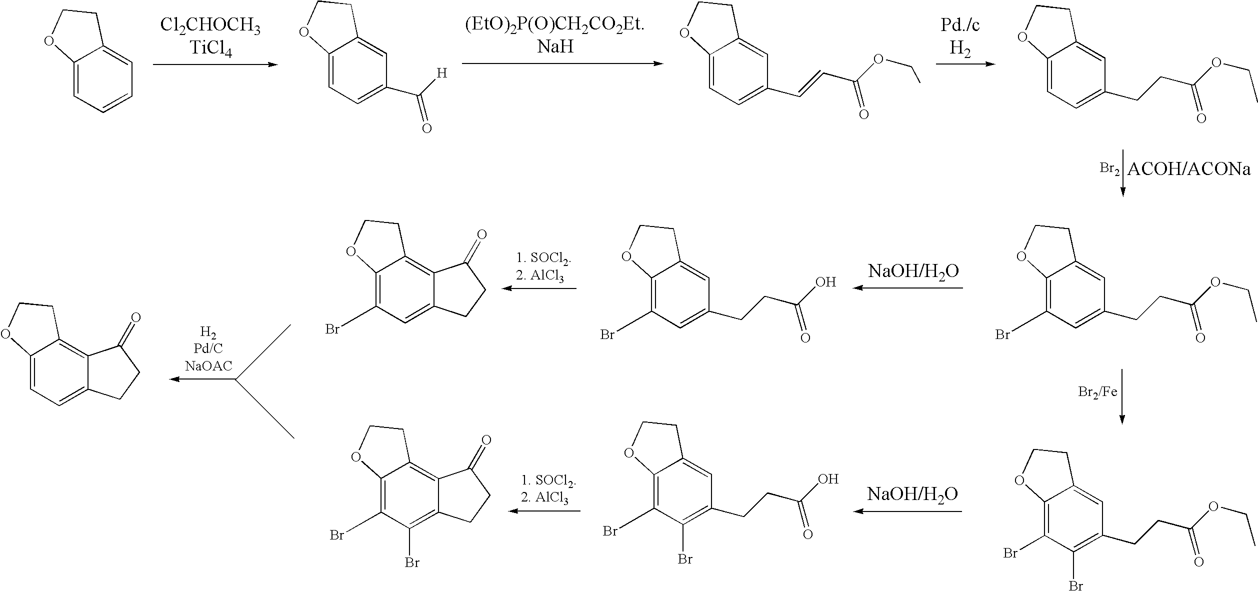

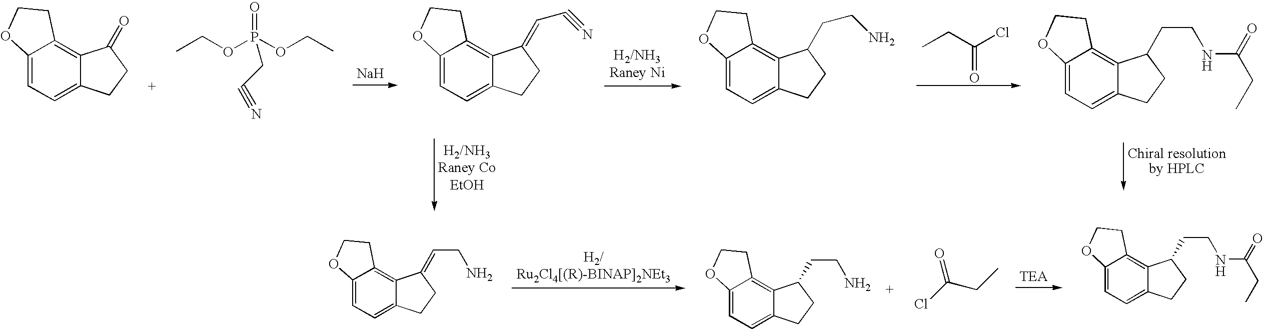

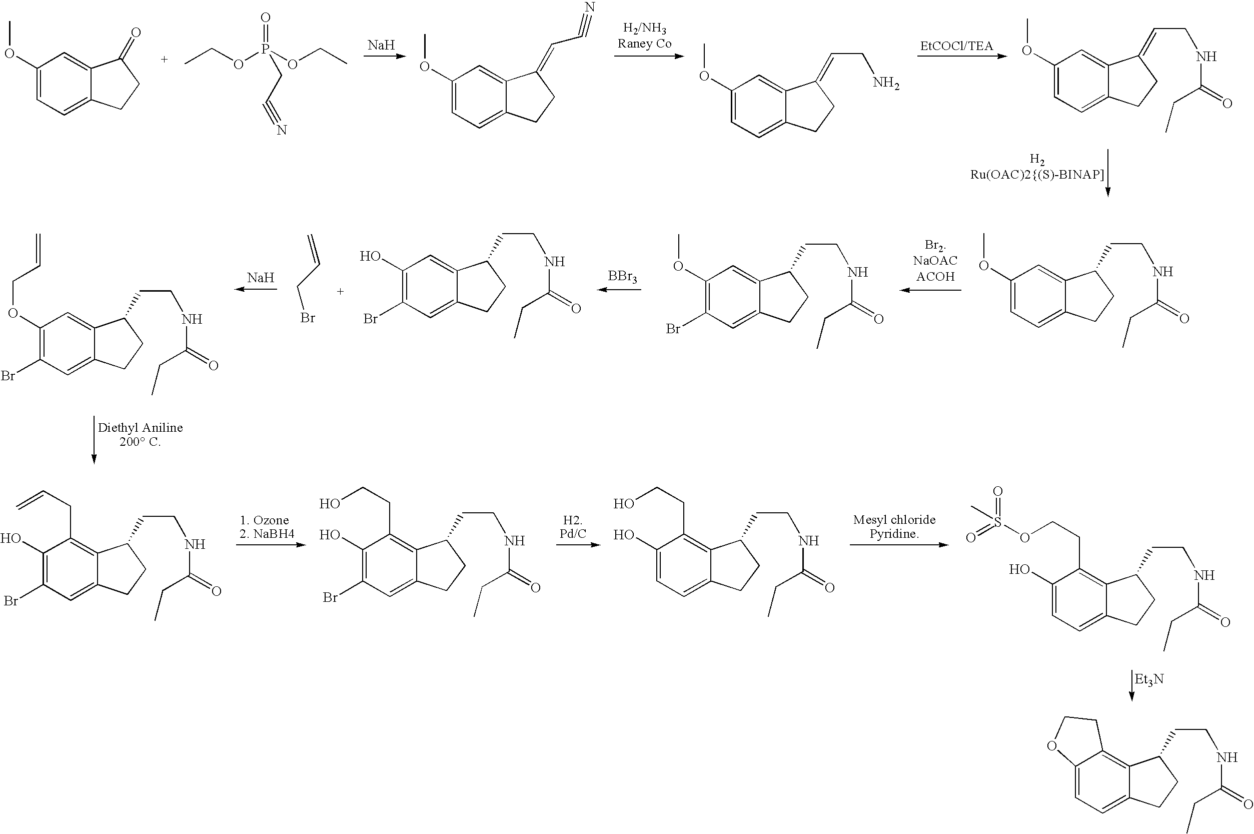

Japan Patent Publication No. 11080106 reports the following process for the preparation of ramelteon:

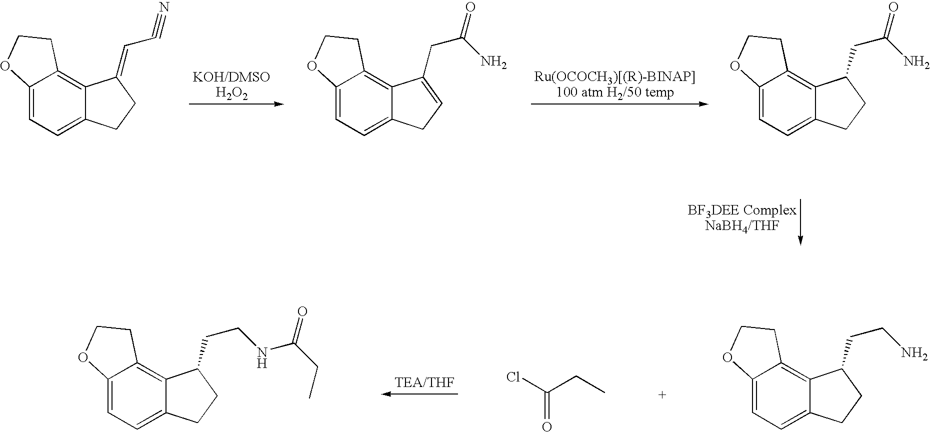

Japan Patent Publication no. 11140073 reports the following process for the preparation of an intermediate of ramelteon:

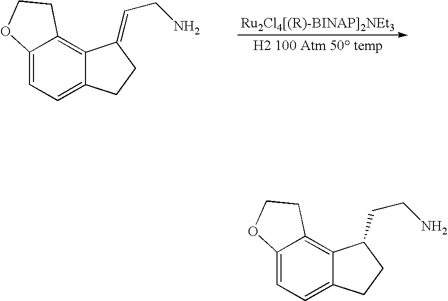

PCT Publication No. WO/2006/030739 reports the following process for the preparation of ramelteon:

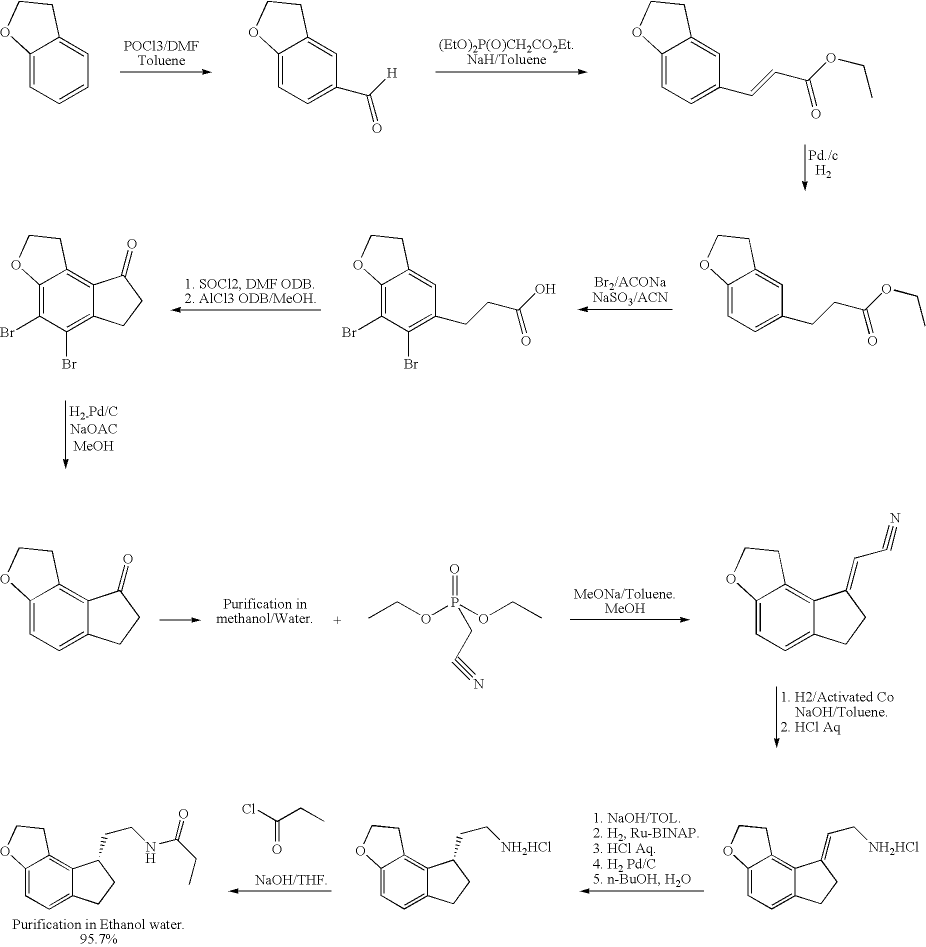

PCT publication No. WO2008/151170 reports the following process for the preparation of ramelteon:

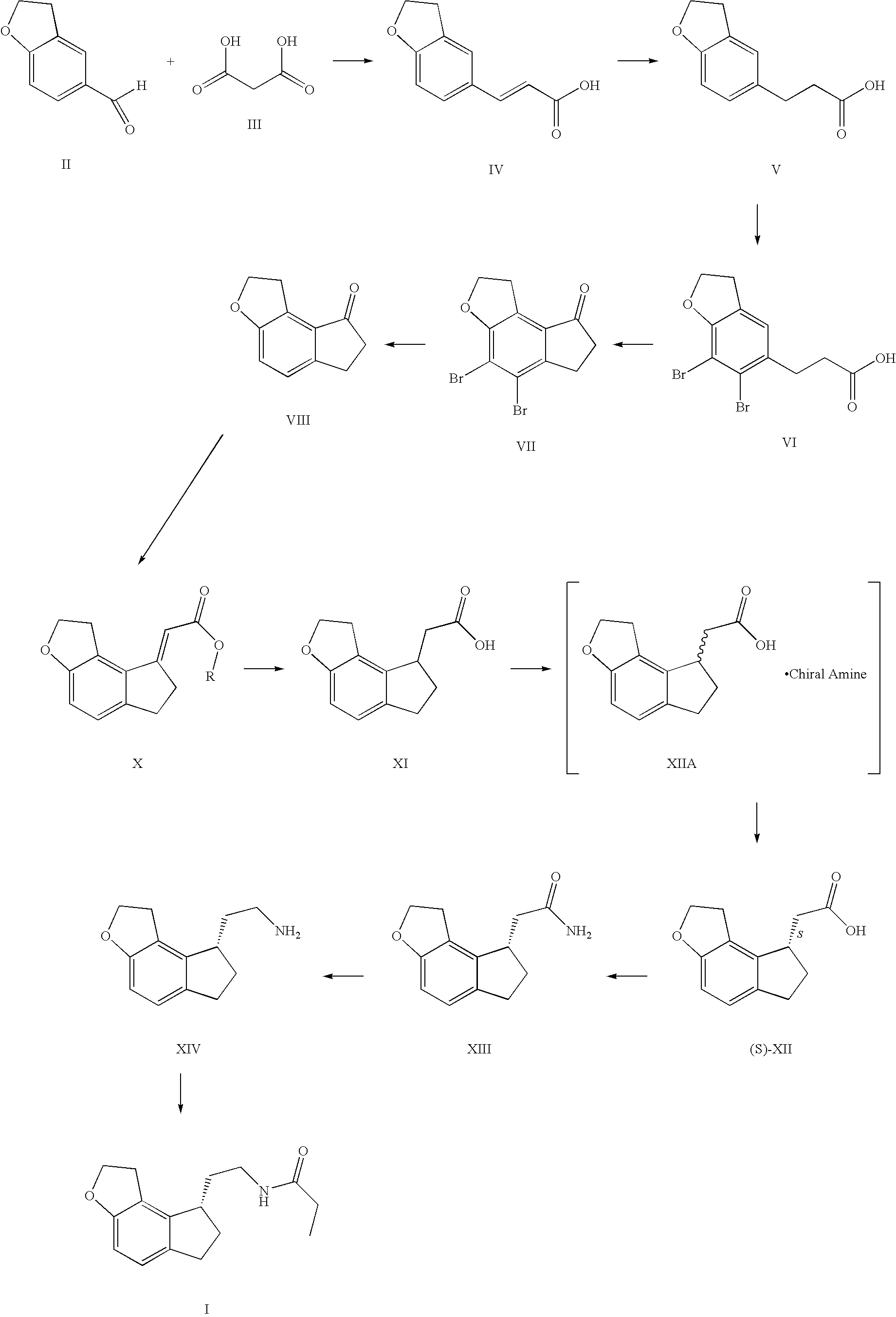

In this process two byproducts a, and b, having the following formulas:

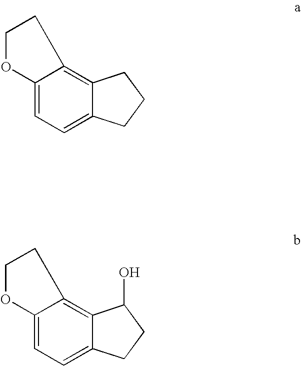

are formed during the dehalogenation step, and have to be removed in order to improve the quality of the desired compound.

There is a pressing need in the art for new low-cost and high-yields processes for the preparation of ramelteon suitable for industrial scale.

### SUMMARY OF THE INVENTION

The present invention provides a method of preparing ramelteon intermediates which proceeds essentially as shown in the following Scheme:

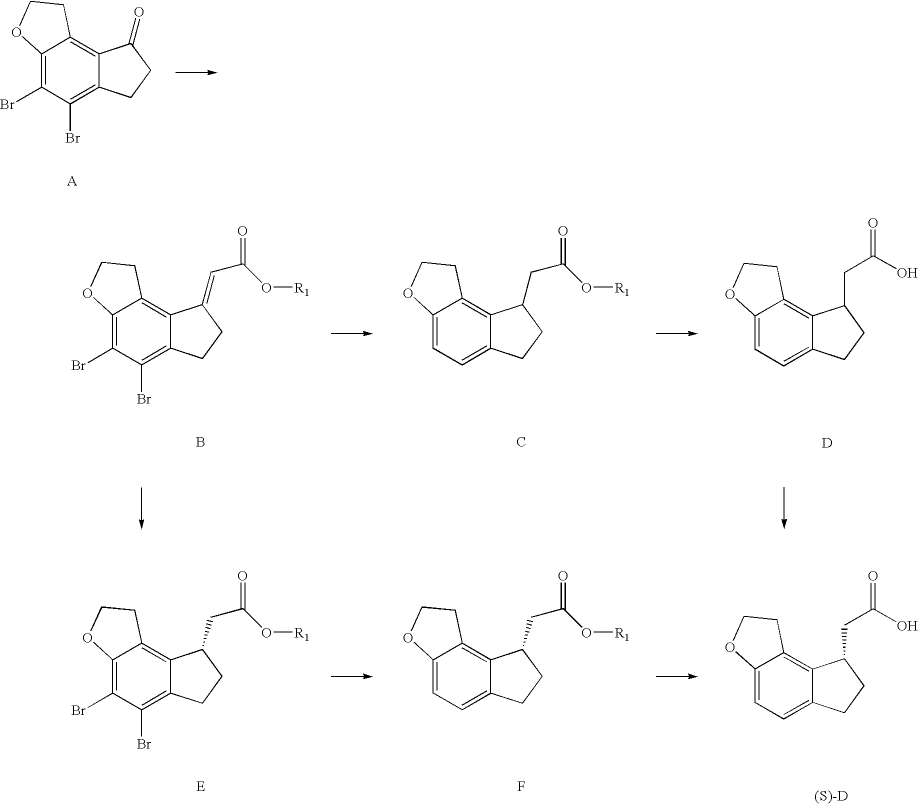

The present invention also provides another method of preparing ramelteon intermediates which proceeds essentially as shown in the following Scheme:

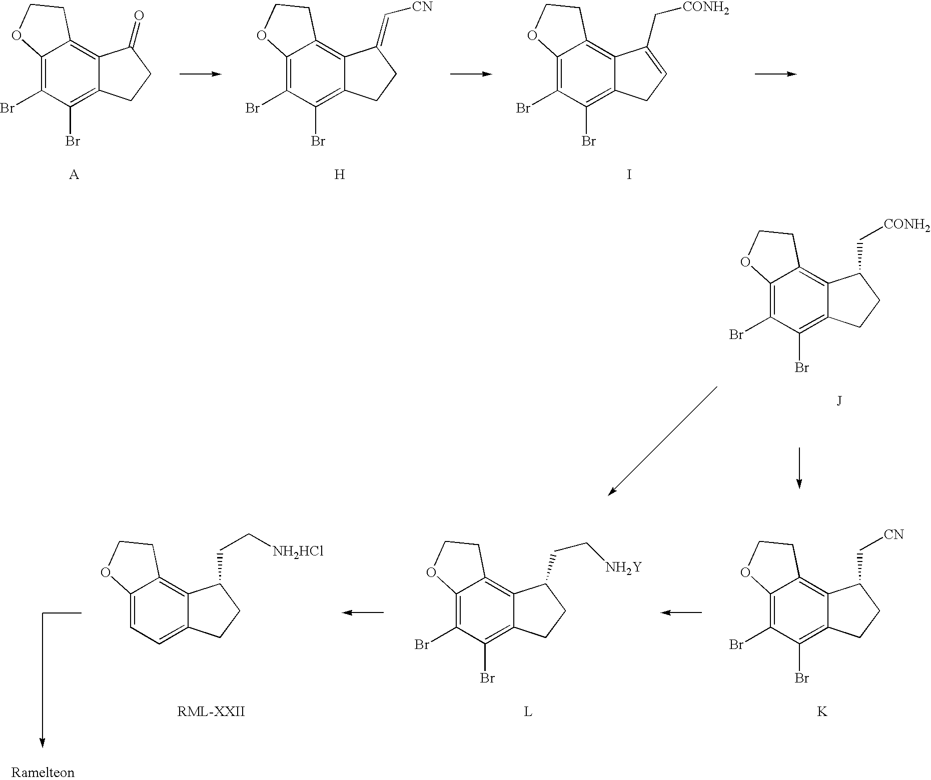

The present invention also provides a stereoselective enzymatic hydrolysis processes for the preparation of compound D, particularly, (S)-D, a key intermediate in the synthesis of ramelteon.

### DETAILED DESCRIPTION OF THE INVENTION

As used herein, a “polar solvent” refers to its ordinary meaning in the art, i.e., solvents with a dielectric constant of less than 15.

As used herein, a “polar protic solvent” refers to its ordinary meaning in the art, i.e., solvent that has a hydrogen atom bound to an oxygen as in a hydroxyl group or a nitrogen as in an amine group. More generally: any molecular solvent which contains dissociable H.

Examples for polar solvents and polar protic solvents are: methanol, ethanol, acetone, ethyl acetate, tetrahydrofuran, isopropanol, n-butanol, and isobutanol.

The present invention provides alternative processes for the preparation of ramelteon and ramelteon intermediates.

These reactions result in a lower cost process for preparing ramelteon in comparison with the processes already described in the prior art. Additionally, they avoid the formation of two byproducts a, and b, having the following formulas:

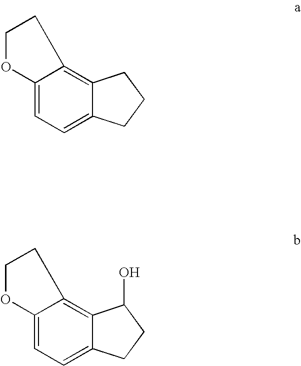

which appear during the dehalogenation step, as described in PCT publication No. WO2008/151170, and have to be removed in order to improve the quality of the desired compound. By avoiding the formation of intermediates a and b, purification is simplified and the overall yield is increased.

The present invention provides a method of preparing ramelteon intermediates which proceeds essentially as shown in the following Scheme:

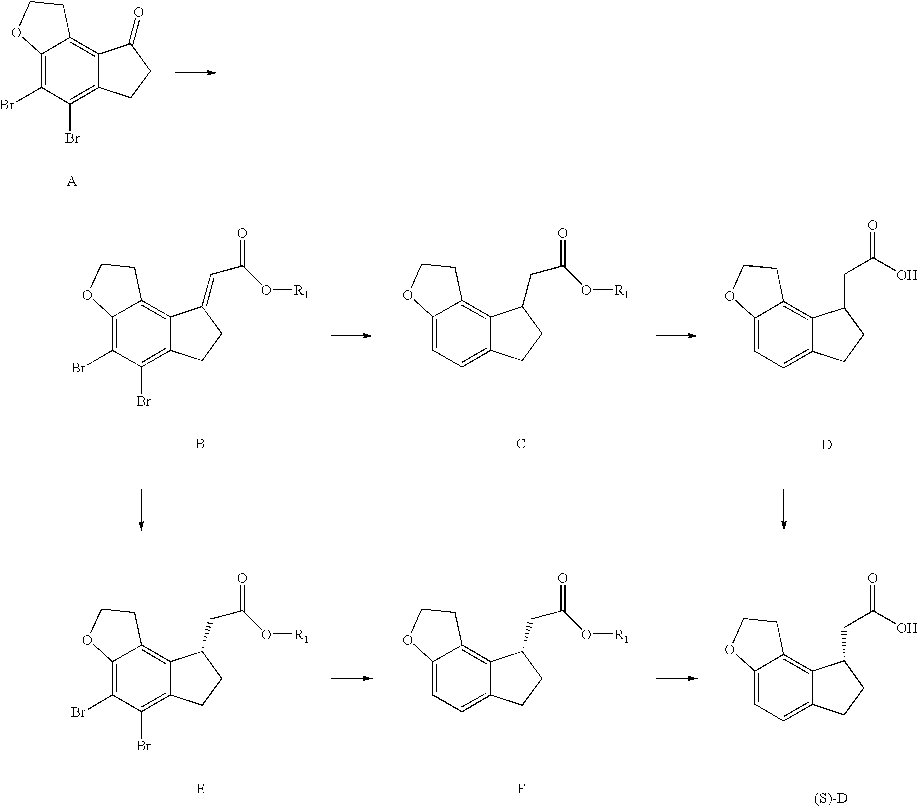

In one embodiment, the present invention encompasses compound B. Compound B can be found in different isomers structures, having the following formulas:

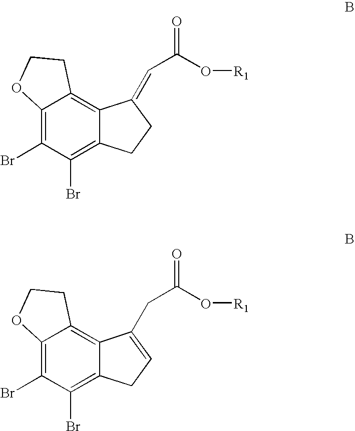

wherein, R₁ is selected from the group consisting of C₁-C₆ straight or branched alkyls, C₆-C₁₀ aryls, and alkylaryl wherein the alkyl contains 1-4 carbons, and the aryl contains 6-12 carbons. Preferably R₁ is C₁-C₆ alkyl, more preferably, C₁-C₃ alkyl, and most preferably methyl or ethyl. R₁ may also preferably be phenyl or benzyl.

As used herein, the term “compound B” refers to the isomers, as mentioned above.

Compound B, having R₁=methyl, can be characterized by an NMR pattern with peaks at about 1.33 to 1.36 (t), 4.22 to 4.27 (q), 6.09 (s), 3.03 to 3.06 (t), 3.32 to 3.36 (t), and 3.46 to 3.50 (t) ppm, as measured in a 400 MHz apparatus, in CDCl₃.

In another embodiment, the present invention encompasses a process for preparing compound of formula B comprising condensing compound of formula A with a trialkylphosphonoacetate. Preferably, the reaction is carried out in the presence of a base. Preferably, the reaction is carried out in the presence of an organic solvent, wherein the organic solvent is preferably selected from the group consisting of C₆-C₁₀ substituted aromatic hydrocarbons, and C₁-C₅ halogenated hydrocarbons. Preferably, the organic solvent is selected from the group consisting of toluene, tetrahydrofuran, dimethylformamide, and dimethylsulfoxide. Preferably, the reaction is carried out under inert atmosphere, such as under nitrogen or argon, preferably nitrogen.

The compound of formula A can be prepared, for example, according to the procedure described at U.S. Pat. No. 6,034,239, WO2006/030739, or WO2008/151170. The compound of formula A is preferably dried prior to the reaction with the trialkylphosphonoacetate, for example, by azeotropic distillation. The reaction is preferably conducted in the absence of water, preferably less than 0.25% water, more preferably less than 0.20%, most preferably less than 0.1% water.

The alkyl groups of the trialkylphosphonoacetate can be the same or different and is preferably selected from the group consisting of C₁-C₆ straight or branched alkyls, C₆-C₁₀ aryls, and arylalkyls wherein the alkyl contains 1-4 carbons, and the aryl contains 6-12 carbons; preferably methyl, ethyl, phenyl, and benzyl.

The base can be selected from the group consisting of one or more of alkali metal hydroxide, metal amides, metal alkoxides, alkyllithiums, amine bases, and alkali metal hydrides. Examples of suitable bases are: sodium hydroxide, potassium hydroxide, sodium hydride, potassium hydride, sodium amide, lithium diisopropylamide, lithium hexamethyldisilazide, sodium methoxide, sodium ethoxide, potassium t-butoxide, BuLi, and 1,8-diazabicyclo[5.4.0]undec-7-ene. Most preferably, the base is selected from the group consisting of sodium methoxide, and sodium hydride.

A solution of sodium methoxide is preferably added drop-wise to a solution of compound of formula A, alkylphosphonoacetate, and the organic solvent. The reaction is carried out at a temperature of about 0° C. to about 250° C., preferably about 50° C. to about 150° C., more preferably about 90° C. to about 100° C.; preferably, for about an hour to about 25 hours, more preferably about 10 hours to about 20 hours, most preferably about 15 hours to about 18 hours.

Preferably, the reaction mixture is quenched with water, and the product is isolated, for example by extraction and distillation. The obtained yield is about 70% to about 90%, typically about 75% to about 87%, more typically about 80% to about 85%.

In another embodiment, the present invention provides compound B having less than 0.01% of any of byproduct a, byproduct b, or combinations thereof, when measured as area by HPLC. Also provided is compound B having less than 0.1% of Compound A, when measured as area by HPLC. In this application, unless specified otherwise, all HPLC purities are percent by area relative to the total area of the HPLC chromatogram (e.g., the total area of compound B and byproducts).

In another embodiment, the present invention encompasses a process for preparing ramelteon comprising obtaining a compound of formula B and further converting it to ramelteon.

In another embodiment, the present invention provides compound C. Compound C may contain less than 0.05% of Compound A or Compound B, as determined by area % HPLC.

In another embodiment, the present invention encompasses a process for preparing compound C by a one pot reaction comprising reducing the double bond in the compound of formula B, and dehalogenation of the bromo groups, wherein either reaction may precede the other. Preferably, the reaction comprises catalytic reduction of the compound of formula B, more preferably, catalytic hydrogenation of compound B.

The reduction and dehalogenation reaction may be carried out by catalytic reduction with hydrogen, preferably, in the presence of sodium acetate, and preferably using Pd—C or Raney-Ni as catalyst. Alternatively, the reduction may be carried out using Zn/HCl or Fe/HCl. Most preferably, the reduction is carried out using Pd—C, preferably, 10% Pd—C. The hydrogen pressure used in the catalytic reduction is preferably in the range of about 0.1 kg/cm² to about 20 kg/cm²; more preferably about 1 kg/cm² to about 10 kg/cm²; and most preferably about 2 kg/cm² to about 5 kg/cm². The reaction is conducted in a solvent selected from the group comprising of C₁ to C₆ halogenated hydrocarbons, C₆ to C₁₄ aromatic hydrocarbons, C₁ to C₅ alcohols, C₂ to C₇ esters, C₄ to C₇ ethers, C₁ to C₅ carboxylic acids, C₅ to C₈ cyclic ethers, water, and suitable mixtures thereof. Preferred solvents are methanol, isopropyl alcohol, dichloromethane, toluene, ethyl acetate, and diethyl ether. Most preferably, the solvent is methanol. When Pd—C or Raney-Ni as catalysts are used, the reaction temperature is generally about 15-70° C.; preferably about 20-60° C.; and the reaction time is generally about 1 hour to about 5 hours; preferably about 1 hour to about 3 hours. When Zn/HCl or Fe/HCl are used, the reaction temperature is generally about 40-60° C.; and the reaction time is generally about 7 hour to about 10 hours. Typically, the amount of catalyst used is about 2-30 g per 100 g of compound B; preferably about 5-20 g per 100 g of compound B; most preferably, about 8-10 g per 100 g of compound B.

The obtained compound of formula C can be further converted to compound of formula D by hydrolyzing under acidic or basic conditions. For example, using an acid selected from the group consisting of sulfuric acid, hydrochloric acid, formic acid, and acetic acid, or by using a base selected from the group consisting of alkali metal hydroxides, metal amides, metal alkoxides, alkyllithiums, amines, and alkali metal hydrides. Most preferably, the conversion is carried out under basic conditions, by using sodium hydroxide.

In another embodiment, the present invention encompasses a process for preparing ramelteon comprising obtaining compound of formula C, as mentioned above, and further converting it to ramelteon.

In another embodiment, the present invention provides compound D. Compound D may contain less than 0.05% of Compound A, Compound B and/or Compound C as determined by Area % HPLC.

Compound D may be further converted to compound (S)-D, for example, according to the procedure that is described in PCT publication No. WO2008/151170. For example, by resolution of the racemic form of compound of formula D by diastereomeric crystallization with an organic chiral amine and acidifying.

Compound (S)-D can be further converted to ramelteon, for example, according to the procedure that is described in PCT publication No. WO2008/151170. For example, by converting the compound of formula (S)-D to an activated acid derivative, followed by ammonolysis of the activated acid derivative, reducing the obtained compound with a reducing agent, and reacting the obtained free base with propionyl chloride to form ramelteon.

In one embodiment, the present invention encompasses compound E, having the following formula:

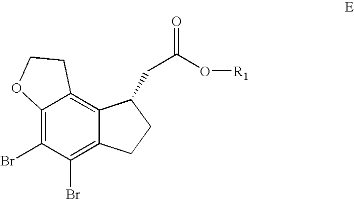

wherein, R₁ is selected from the group consisting of C₁-C₆ straight or branched alkyls, C₆-C₁₀ aryls, and alkylaryl wherein the alkyl contains 1-4 carbons, and the aryl contains 6-12 carbons. Preferably R₁ is C₁-C₆ alkyl, more preferably, C₁-C₃ alkyl, and most preferably methyl or ethyl. R₁ may also preferably be phenyl or benzyl.

In another embodiment, the present invention encompasses a process for preparing compound E comprising stereoselective reduction of the double bond of compound B.

Stereoselective reduction can be carried out by using an enantiomerically pure transition metal catalyst under hydrogen pressure, preferably in the presence of a polar solvent. The enantiomerically pure catalyst is preferably selected from the group consisting of (+)-Ru(OAc)₂[(R)—BINAP], (+)-Ru(Cl₂)benzene-[(R)—BINAP], and (+)-Ru(Cl₂)p-cymene-[(R)—BINAP], with (+)-Ru(OAc)₂[(R)—BINAP]; most preferably, (+)-Ru(OAc)₂[(R)—BINAP]. The enantiomeric purity is preferably above about 99.5%.

In catalytic reduction, the hydrogen pressure may preferably be in the range of 0.1 to 100 kg/cm²; preferably 5-10 kg/cm². Preferably, the reaction is carried out at a temperature between 10° C. and 50° C., more preferably at about 25° C. The reaction is preferably conducted in any suitable solvent, which may for example be selected from the group consisting of C₁-C₆ halogenated hydrocarbons, C₆ to C₁₄ aromatic hydrocarbons, C₁ to C₅ alcohols, C₂ to C₇ esters, C₄ to C₇ ethers, C₁ to C₅ carboxylic acids, water, or suitable mixtures of these solvents. Preferred solvents are water, methanol, isopropyl alcohol, dichloromethane, toluene, ethyl acetate, and diethyl ether.

In another embodiment, the present invention provides compound E. Compound C may contain less than 0.05% of Compound A or Compound B as determined by Area % HPLC.

In another embodiment, the present invention encompasses a process for preparing ramelteon comprising obtaining compound of formula E and further converting it to ramelteon.

In one embodiment, the present invention encompasses compound F, having the following formula:

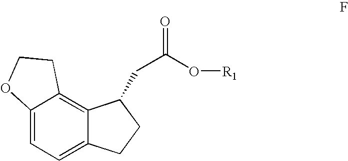

wherein R₁ is selected from the group consisting of C₁-C₆ straight or branched alkyls, C₆-C₁₀ aryls, and alkylaryl, wherein the alkyl contains 1-4 carbons, and the aryl contains 6-12 carbons. Preferably R₁ is C₁-C₆ alkyl, more preferably, C₁-C₃ alkyl, and most preferably methyl or ethyl. R₁ may also preferably be phenyl or benzyl.

In another embodiment, the present invention encompasses a process for preparing compound F comprising dehalogenation of compound E. Preferably, the process comprises catalytic hydrogenation of compound E.

Preferably, the reaction is carried out in the presence of sodium acetate, Pd/C, and acetic acid under a hydrogen atmosphere.

The hydrogen pressure may preferably be in the range of 0.1 to 100 kg/cm²; preferably 5-10 kg/cm²; most preferably 2-3 kg/cm².

In another embodiment, the present invention encompasses a process for preparing ramelteon comprising obtaining compound of formula F and further converting it to ramelteon.

In another embodiment, the present invention encompasses a process for preparing ramelteon comprising:

a) condensing compound of formula A with a trialkylphosphonoacetate, preferably wherein the reaction is carried out in the presence of a base, and an organic solvent preferably selected from the group consisting of C₆-C₁₀ substituted aromatic hydrocarbons, and C₁-C₅ halogenated hydrocarbons to obtain the compound of formula B;

b) reducing the obtained compound of formula B, and dehalogenation of the bromo groups to obtain the compound of formula C;

c) hydrolyzing the obtained compound of formula C to obtain compound of formula D; and

d) further converting it to ramelteon, for example, according to the procedure that is described in PCT publication No. WO2008/151170.

Preferably, steps a, b, c and d are defined in any of the above passages.

In another embodiment, the present invention encompasses another process for preparing ramelteon comprising:

a) condensing compound of formula A with a trialkylphosphonoacetate, preferably wherein the reaction is carried out in the presence of a base, and an organic solvent preferably selected from the group consisting of C₆-C₁₀ substituted aromatic hydrocarbons, and C₁-C₅ halogenated hydrocarbons to obtain the compound of formula B;

b) reducing the obtained compound of formula B in a stereoselective manner to obtain the compound of formula E;

c) dehalogenating the “bromo” groups of compound of formula E to obtain the compound formula F; and

d) further converting it to ramelteon. For example, according to the procedure that is described in PCT publication No. WO2008/151170.

Preferably, steps b and c are conducted in one step by a stereoselective catalytic hydrogenation reaction. Preferably, steps a, b, c and d are defined in any of the above passages.

The present invention also provides a method of preparing ramelteon intermediates which proceeds essentially as shown in the following Scheme:

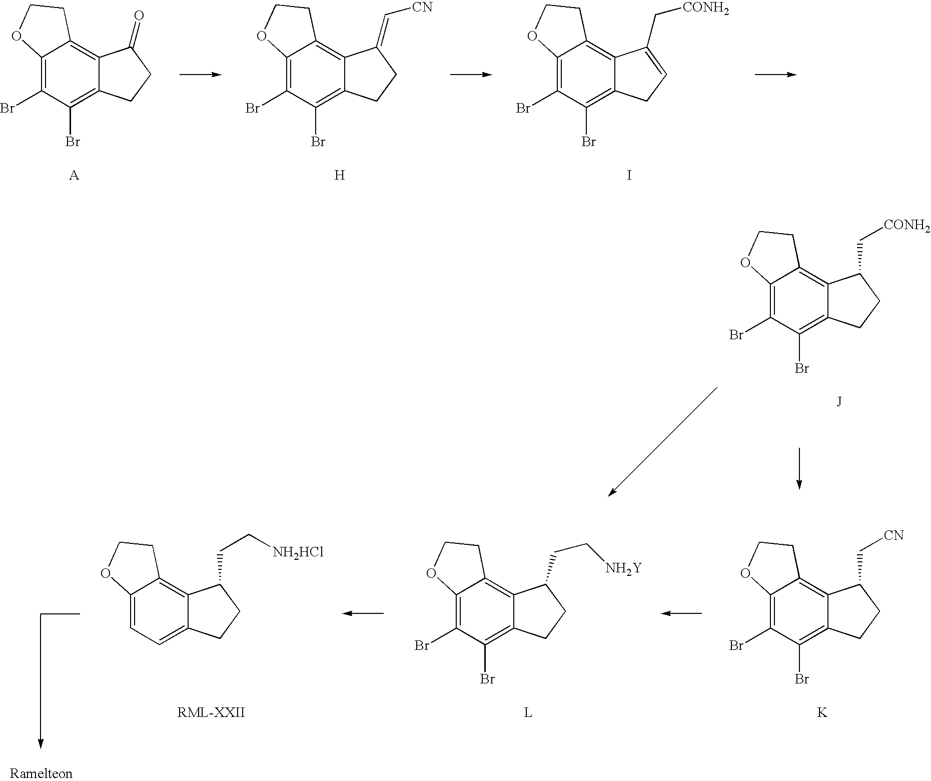

In one embodiment, the present invention encompasses compound H, having the following formula:

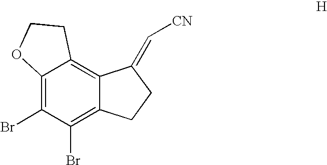

Compound H can be characterized by an NMR pattern with peaks at about 5.46 (s, 1H), 4.79-4.84 (t, 2H), 3.37-3.4 (t, 2H), and 4.07-3.17 (m, 4H), as measured in a 400 MHz apparatus, in CDCl₃.

Compound H may contain less than 0.1% of Compound A as measured by area HPLC.

In another embodiment, the present invention encompasses a process for preparing the compound of formula H comprising condensing the compound of formula A with a dialkyl cyanomethyl phosphonate. The reaction is preferably conducted in the presence of a base. The reaction is preferably conducted in the presence of an organic solvent.

The alkyl groups of the dialkyl cyanomethyl phosphonate can be the same or different (preferably the same) selected from the group consisting of C₁-C₆ straight or branched alkyls, C₆-C₁₀ aryls, and alkylaryl, wherein the alkyl contains 1-4 carbons, and the aryl contains 6-12 carbons. Preferably the groups are methyl, ethyl, phenyl, and benzyl.

The organic solvent is selected from the group consisting of C₆-C₁₀ substituted aromatic hydrocarbons, C₄-C₈ cyclic ethers and C₃-C₈ acyclic ethers, and C₁-C₅ halogenated hydrocarbons. Most preferably, the organic solvent is toluene, dimethylformamide, tetrahydrofuran, and dimethylsulfoxide. Typically, the reaction is carried out using an azeotropic distillation, or under inert atmosphere.

The base is preferably selected from the group consisting of alkali metal hydroxides, metal amides, metal alkoxides, alkyllithiums, amine bases, and alkali metal hydrides. Examples of suitable base are: sodium hydroxide, potassium hydroxide, sodium hydride, potassium hydride, sodium amide, lithium diisopropylamide, lithium hexamethyldisilazide, sodium methoxide, sodium ethoxide, potassium t-butoxide, BuLi, and 1,8-diazabicyclo[5.4.0]undec-7-ene. Most preferably, the base is sodium methoxide.

A solution of sodium methoxide is preferably added dropwise to the solution of compound of formula A and dialkyl cyanomethyl phosphonate in toluene. The reaction is preferably carried out at a temperature of about 0° C. to about 20° C., preferably about 0° C. to about 10° C., more preferably about 0° C. to about 5° C.; for about an hour to about 8 hours, preferably about an hour to about 5 hours, most preferably about an hour to about 3 hours.

Preferably, the reaction mixture is quenched with water, and the product is isolated, for example by extraction, and distillation.

In another embodiment, the present invention encompasses a process for preparing ramelteon comprising obtaining compound of formula H and further converting it to ramelteon.

In another embodiment, the present invention encompasses compound I. Compound I can be found in different isomer structures, having the following formulas:

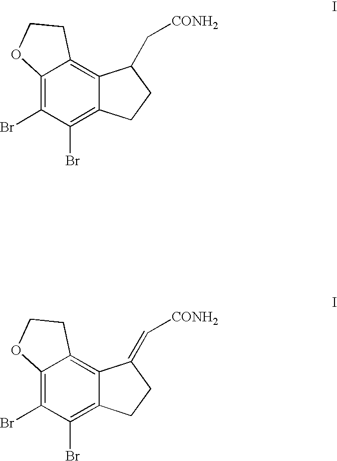

Preferably the compound of formula I is isolated. Compound I may contain less than 0.05% of Compound A and/or Compound H as measured by area HPLC.

In a specific embodiment, compound I, having the following formula:

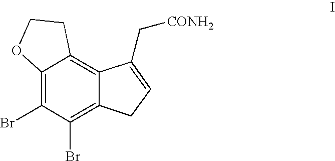

can be characterized by an NMR pattern with peaks at about 7.05 (s, 1H), 6.15 (s, 1H), 6.08 (s, 1H), 4.66-4.71 (t, 2H), 3.38-3.42 (t, 2H), 3.23-3.26 (m, 2H), 2.88-2.91 (t, 2H), as measured in a 400 MHz apparatus, in CDCl₃.

As used herein, the term “compound “I” refers to the isomers, as mentioned above.

In another embodiment, the present invention encompasses a process for preparing compound of formula I comprising hydrolyzing the compound of formula H.

The hydrolysis reaction may be carried out by means of various nitrile hydrolysis reactions known in the art. It is preferably carried out via the Radziszewski reaction, wherein hydrogen peroxide is added to an alkaline solution of compound H in a mixed organic/aqueous solvent mixture; and maintaining the mixture for sufficient time to obtain compound of formula I. Suitable organic solvents include, polar protic solvents, and can include, but are not limited to, dimethylsulfoxide, dimethylformamide, and dimethylacetamide.

The inorganic base is preferably potassium hydroxide. Preferably, the hydrogen peroxide is added as a 30% solution of hydrogen peroxide and water. Preferably, the solution is added drop-wise. The reaction mixture is maintained at about 20° C. to about 50° C., preferably at about 25° C. to about 35° C., most preferably at about room temperature, for about an hour to about 10 hours, preferably about 2 hours to about 5 hours, most preferably about 3 hours to about 4 hours.

In another embodiment, the present invention encompasses a process for preparing ramelteon comprising obtaining the compound of formula I, and further converting it to ramelteon.

In another embodiment, the present invention encompasses compound J, in racemic form, as an isolated enantiomer, or a mixture thereof having the following formula:

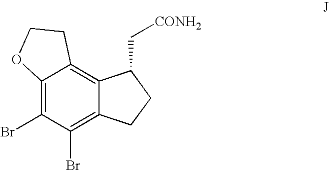

The compound J may contain less than 0.05% of one or more of Compound A, Compound H, and/or Compound I, as measured by area HPLC.

Preferably the compound of formula J is isolated. Preferably compound J contains less than 10% of the (R) enantiomer, more preferably, less than 1% of the (R) enantiomer.

In another embodiment, the present invention encompasses a process for preparing the ramelteon intermediate of formula J, in racemic form, as an isolated enantiomer, or as a mixture thereof, comprising reducing the compound of formula I. Preferably, the reduction reaction is an asymmetric reduction. The asymmetric reduction is preferably catalytic. Preferably the catalyst is a chiral ruthenium catalyst, such as Ru(OAc)₂[(R)—BINAP], Ru(Cl₂)benzene-[(R)—BINAP], and Ru(Cl₂)p-cymene-[(R)—BINAP], with Ru(OAc)₂[(R)—BINAP] being preferred.

The reduction is preferably carried out by forming a mixture of compound of formula I, an enantiomerically pure ruthenium catalyst, and a polar protic solvent, in the presence of a hydrogen source. Preferably, Ru(OAc)₂[(R)—BINAP] is used, and the obtained compound J is (S)-J.

Suitable ruthenium catalyst include, but are not limited to, Ru(OAc)₂-[(R)—BINAP], Ru(Cl₂)benzene-[(R)—BINAP], and Ru(Cl₂)p-cymene-[(R)—BINAP]; preferably the catalyst is Ru(OAc)₂[(R)—BINAP].

Preferably, the polar protic solvent is a C₁-C₅ alcohol, more preferably ethanol.

The hydrogen pressure may preferably be in the range of 1 kg/cm² to 20 kg/cm², preferably about 5 kg/cm² to about 15 kg/cm², most preferably about 5 to about 10 kg/cm². The reaction mixture is preferably maintained at a temperature of about 25° C. to about 80° C., preferably about 25° C. to about 50° C., most preferably about 50° C.

In another embodiment, the present invention encompasses a process for preparing ramelteon comprising obtaining compound of formula J, in racemic form, as an isolated enantiomer, or as a mixture thereof, and further converting it to ramelteon.

In another embodiment, the present invention encompasses compound K, in racemic form, as an isolated enantiomer, or a mixture thereof, having the following formula:

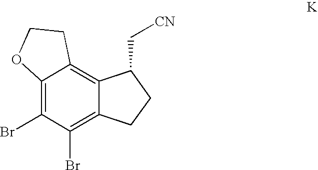

Preferably the compound of formula K is isolated. Preferably compound K contains less than 10% of the (R) enantiomer, preferably, less than 5% of the (R) enantiomer. The compound K may contain less than 0.05% of one or more of Compound A, Compound H, Compound I and/or Compound J, as measured by area HPLC.

In another embodiment, the present invention encompasses a process for preparing compound of formula K, in racemic form, as an isolated enantiomer, or a mixture thereof, comprising dehydrating the amide group of compound of formula J to obtain compound of formula K. Preferably, (S)-J is used, and the obtained compound is (S)-K.

The dehydration is carried out by using a dehydrating reagent preferably in the presence of an organic solvent. The dehydrating reagent can be selected from the group consisting of such as P₂O₅, POCl₃, and SOCl₂. The reaction mixture is preferably, heated to about 60° C. to about 100° C. More preferably the reaction is heated to about 80° C. to about 85° C. Preferably, the reaction is heated for about 3 hours to about 8 hours, more preferably, for about 4 hours to about 5 hours. The reaction mixture can then be quenched with ice water, and the obtained compound K may be recovered for example by extraction and distillation.

The organic solvent can be preferably selected for the group consisting of C₆-C₁₀ substituted aromatic hydrocarbons, C₅-C₆ aliphatic hydrocarbons, and C₁-C₅ halogenated hydrocarbons. Preferably the organic solvent is toluene.

In another embodiment, the present invention encompasses another process for preparing compound K comprising asymmetric reduction of compound H.

The asymmetric reduction is preferably catalytic. Preferably the catalyst is a chiral transition metal catalyst. The catalyst is preferably based on ruthenium, rhodium, iridium, and the like. Most preferably, the catalyst is a ruthenium catalyst. Suitable ruthenium catalyst include, but are not limited to, Ru(OAc)₂-[(R)—BINAP], Ru(Cl₂)benzene-[(R)—BINAP], Ru(Cl₂)p-cymene-[(R)—BINAP], RuBr₂(p-Cymene)[(R)—BINAP], RuI₂(p-Cymene)[(R)—BINAP]; preferably the catalyst is Ru(OAc)₂-[(R)—BINAP].

The reduction is preferably carried out by forming a mixture of compound H, an enantiomerically pure ruthenium catalyst, and a polar protic solvent, in the presence of a hydrogen source.

Preferably, the polar protic solvent is selected from the group consisting of a C₁-C₅ alcohol (such as methanol, ethanol, isopropanol, butanol, and tert-butanol), acetonitrile, water, toluene, and mixture thereof. Most preferably, the solvent is ethanol.

The hydrogen pressure may preferably be in the range of 2 kg/cm² to 25 kg/cm², preferably about 5 kg/cm² to about 15 kg/cm², most preferably about 5 to about 10 kg/cm². The reaction mixture is preferably maintained at a temperature of about 25° C. to about 100° C., preferably about 25° C. to about 50° C., most preferably about 50° C.

In another embodiment, the present invention encompasses a process for preparing ramelteon comprising obtaining the compound of formula K, in racemic form, as an isolated enantiomer, or as a mixture thereof, and further converting it to ramelteon.

In another embodiment, the present invention encompasses a process for preparing the compound of formula L, comprising reducing the cyano functional group of compound K.

The reaction is preferably carried out by catalytic hydrogenation. Preferably the catalyst is Raney nickel or Raney cobalt. Preferably the reaction is carried out in the presence of a solvent, preferably selected from the group consisting of C₆-C₁₂ aromatic hydrocarbons, and C₁-C₄ alcohols, more preferably methanol, ethanol, and toluene. In a particularly preferred embodiment the reaction is carried out by forming a mixture of compound K, acetonitrile, toluene, and a polar organic solvent; and adding Raney nickel or Raney cobalt, and a base under hydrogen pressure.

The polar organic solvent is preferably selected from the group consisting of C₁-C₅ alcohols. Preferably, the polar organic solvent is methanol. The base is preferably selected from the group consisting of one or more of sodium hydroxide, potassium hydroxide, sodium carbonate, potassium carbonate, cesium carbonate, sodium bicarbonate, potassium bicarbonate, aromatic amines such as pyridine, and lutidine, tertiary amines such as triethyl amine, tripropyl amine, tributyl amine, cyclohexyl dimethyl amine, N-methylpiperidine, N-methylpyrrolidine, and N-methylmorpholine. Preferably, the base is sodium hydroxide, potassium hydroxide, sodium carbonate, or potassium carbonate. Most preferably, the base is potassium hydroxide.

Typically, the reaction mixture is preferably maintained at a temperature of about 20° C. to about 80° C., preferably about 40° C. to about 70° C., and most preferably about 35° C. to about 55° C., preferably for about 5 hours to about 20 hours, more preferably about 10 hours to about 15 hours, and most preferably about 10 hours to about 12 hours.

The hydrogen pressure may preferably be in the range of about 1 kg/cm² to about 20 kg/cm², preferably about 1 kg/cm² to about 10 kg/cm², and most preferably about 3 kg/cm² to about 5 kg/cm².

In another embodiment, the present invention encompasses another process for preparing the compound of formula L, comprising reduction of compound J with an amide reducing agent.

Preferably, the amide reducing agent can be, for example borane, sodium borohydride in presence of boron-trifluoride diethyl ether complex in tetrahydrofuran, or an aluminum hydride such as LiAlH₄, sodium bis(2-methoxyethoxy)aluminum hydride (Red-Al™) or diisobutylaluminum hydride. The reaction is carried out at about −20° C. to 50° C.

In another embodiment, the present invention encompasses a process for preparing ramelteon comprising obtaining compound of formula L, of the following formula:

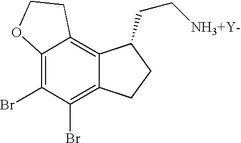

in racemic form, as an isolated enantiomer, or a mixture thereof, and further converting it to ramelteon.

In another embodiment, the present invention encompasses a process for preparing ramelteon comprising:

(a) condensing compound of formula A with a dialkyl cyanomethyl phosphonate to obtain compound H;

(b) hydrolyzing the compound of formula H to obtain compound I;

(c) asymmetrically reducing compound Ito obtain compound J;

(d) dehydrating the amide group of compound of formula J to obtain compound of formula K;

(e) reducing the cyano functional group of compound K to obtain the compound of formula L; and

(f) converting the compound of formula L to ramelteon.

Preferably step a of the reaction is conducted in the presence pf a base, in toluene under inert atmosphere. Steps a, b, c, d, e and f may be carried out in accordance with any of the embodiments and preferred embodiments discussed above.

In another embodiment, the present invention encompasses a process for preparing ramelteon comprising:

(a) condensing compound of formula A with a dialkyl cyanomethyl phosphonate to obtain compound H;

(b) hydrolyzing the compound of formula H to obtain compound I;

(c) asymmetrically reducing compound Ito obtain compound J;

(d) reducing compound J with an amide reducing agent to obtain compound L; and

(e) converting the compound of formula L to ramelteon.

In another embodiment, the present invention encompasses an alternative route for preparing ramelteon, which proceeds essentially as shown in the following

Scheme:

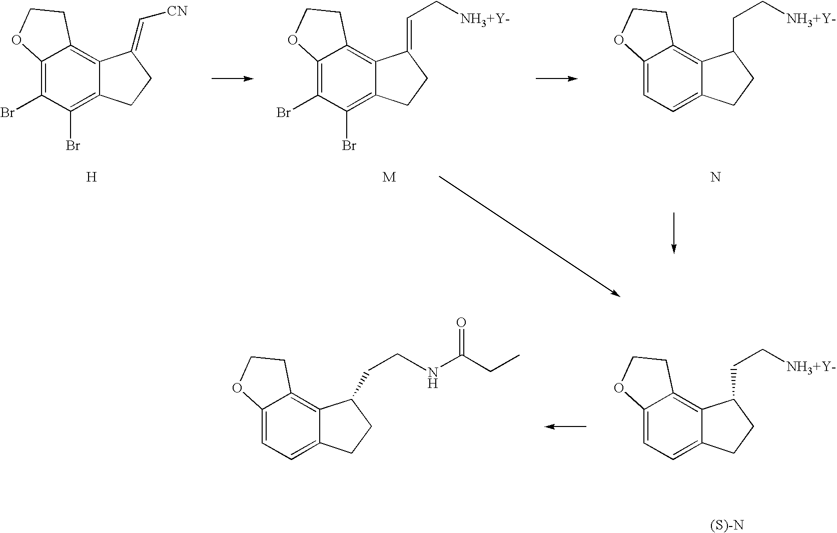

In another embodiment, the present invention encompasses compound M, having the following formula:

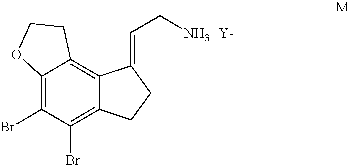

wherein Y⁻ is an anion, preferably a pharmaceutical acceptable anion such as oxalate, sulphate, nitrate, phosphate, perchlorate, borate, halide, acetate, trifluoroacetate, tartrate, maleate, citrate, fumarate, succinate, palmoate, methanesulphonate, benzoate, salicylate, benzenesulfonate, ascorbate, glycerol phosphate, or ketoglutarate.

In another embodiment, the present invention encompasses a process for preparing compound M comprising reducing the cyano functional group of compound H. preferably the reduction is achieved by catalytic hydrogenation. More preferably the catalyst is H₂/Raney-Co.

In catalytic hydrogenations, the hydrogen pressure is preferably about 1 kg/cm² to about 20 kg/cm²; preferably about 1-10 kg/cm²; most preferably about 3-5 kg/cm². The reaction is conducted in a solvent selected from the group comprising of one or more of C₆ to C₁₄ aromatic hydrocarbons, Cᵢ to C₅ alcohols, C₂ to C₇ esters, Cᵢ to C₅ carboxylic acids, C₂ to C₆ ethers, water, or suitable mixtures thereof; preferably methanol, isopropyl alcohol, toluene, ethyl acetate, or diethyl ether. The reaction temperature is generally about 20-80° C.; preferably about 40-70° C.; most preferably 50-55° C.

In another embodiment, the present invention encompasses a process for preparing ramelteon comprising obtaining compound M and further converting it ramelteon.

In another embodiment, the present invention encompasses a process for preparing compound (S)—N, of the following formula:

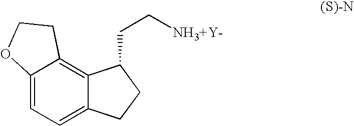

wherein Y⁻ is an anion, preferably a pharmaceutical acceptable anion such as oxalate, sulphate, nitrate, phosphate, perchlorate, borate, halide, acetate, trifluoroacetate, tartrate, maleate, citrate, fumarate, succinate, palmoate, methanesulphonate, benzoate, salicylate, benzenesulfonate, ascorbate, glycerol phosphate, or ketoglutarate; comprising reduction of the double bond of compound M, and dehalogenating of the bromo functional groups to obtain compound (S)—N. Preferably, the process comprises catalytic hydrogenation of the compound of formula M. More preferably the catalyst is Pd/C or Raney Ni. Alternatively, a further preferred method is by reduction, preferably using Zn/HCl, or Fe/HCl.

Thus, the reaction can be carried out, for example by reduction with H₂/Pd—C, H₂/Raney-Ni, Zn/HCl, or Fe/HCl. The preferred method is catalytic reduction with hydrogen, more preferably, in the presence of sodium acetate or potassium acetate and 10% Pd—C catalyst. Preferably, the reaction is carried out in the presence of an organic solvent, selected from the group consisting of C₁-C₄ alcohols, preferably, methanol, or ethanol. In catalytic hydrogenations, the hydrogen pressure is preferably about 0.1 kg/cm² to 20 kg/cm², more preferably about 1-10 kg/cm², and most preferably 2-5 kg/cm². The reaction is preferably maintained at a temperature of about room temperature to about 80° C., preferably about 40° C. to about 70° C., and most preferably about 50° C. to about 55° C.

In another embodiment, the present invention encompasses a process for preparing ramelteon comprising obtaining compound (S)—N and further converting it to ramelteon.

In another embodiment, the present invention encompasses a process for preparing ramelteon comprising:

(a) reducing the cyano functional group of compound H using H₂/Raney-Co to obtain compound M;

(b) dehalogenation of the bromo functional groups of compound M to obtain compound (S)—N; wherein compound N may or may not be isolated and (c) converting the compound of formula (S)-N to ramelteon.

In another embodiment, the invention is directed to a process for the preparation of compound D, of the following formula:

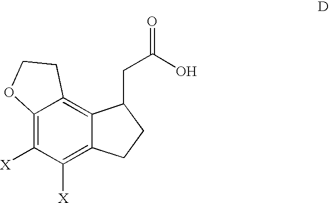

wherein X is Br or H, particularly, the ramelteon intermediate (S)-D, via enzymatic hydrolysis of compound C, and more preferably, compound C(i), of the following formulas:

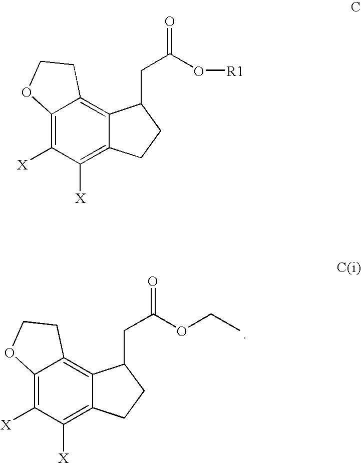

The process comprises an enzymatic hydrolysis of compound C, and more preferably, compound C(i) for the preparation of (S)-D of the invention of high enantiomeric purity.

Compound C, and particularly, compound C(i) can be prepared for example, according to the procedure disclosed in PCT Publication No. WO2008/151170, or by the processes described above.

The process of the invention for the preparation of the ramelteon intermediate (S)-D of the formula comprises combining compound C, more preferably, compound C(i) with an enzyme that hydrolyzes an ester to an acid in a stereoselective manner to obtain a reaction mixture, and maintaining the reaction mixture to obtain the intermediate. The enzyme can be isolated from a natural source or synthesized with recombinant technology.

Preferably, the enzyme is one that is capable of producing (S)-D with a d.e. of about 90% or higher in the processes of the invention. Preferably, the enzyme is one that capable of producing (S)-D with a yield of about 50% of theoretical or higher in the processes of the invention.

Preferably, the enzyme is a hydrolase.

As used herein, “hydrolase” refers to an enzyme that catalyzes the hydrolysis of a chemical bond in a stereoselective manner, optionally with the aid of co-factor. Hydrolases are commercially available, for example, from Codexis, Inc. under the catalog numbers NZL-102-LYO, NZL-103-LYO, NZL-107-LYO.

In one embodiment, the present invention provides a process for preparing (S)- or (R)-D, of the following formula:

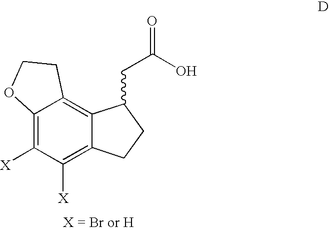

comprising combining compound C, more preferably, compound C(i) of formula:

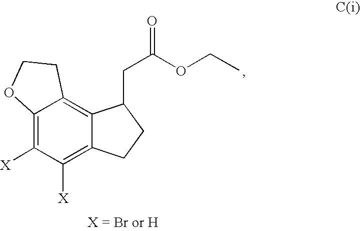

an enzyme that stereoselectively hydrolyzes an ester to form an acid, and a co-factor, to obtain a reaction mixture; and maintaining the mixture to obtain (S)- or (R)-compound D.

In another embodiment, the present invention provides a process for preparing compound D comprising forming a solution comprising compound C, and more preferably, compound C(i), an enzyme selected from the group consisting of NZL-102-LYO, NZL-103-LYO, and NZL-107-LYO; and maintaining the solution, preferably with stirring, for a time sufficient to convert compound C, or C(i) to compound D by enzymatic hydrolysis.

In one embodiment, the present invention further provides a process for preparing ramelteon. The process comprises preparing (S)-D by the enzymatic hydrolysis process of the invention, and converting the (S)-D into ramelteon. NZL enzymes are commercially available. Examples of these include NZL-102-LYO, NZL-103-LYO, and NZL-107-LYO.

Preferably, the enzyme is isolated. The enzyme can be separated from any host, such as mammals, filamentous fungi, yeasts, and bacteria. The isolation, purification, and characterization of a NZL enzyme is described in, for example, Electronic Journal of Biotechnology, 2006, vol 9(1), 69-85. The enzyme is preferably prepared by recombinant means. Preferably, the enzyme is purified, preferably with a purity of about 90% or more, more preferably with a purity of about 95% or more. Preferably, the enzyme is substantially cell-free. Most preferably, the enzyme is a lyophilized preparation, such as are formulated by BioCatalytics Inc., Pasadena, Calif.

Optionally, the reaction is carried out in the presence of a co-factor.

As used herein, the term “co-factor” refers to an organic compound that operates in combination with an enzyme which catalyzes the reaction of interest. Co-factors include, for example, NAD⁺ and NADP⁺, coenzyme A, tetrahydrofolic acid, menaquinone, ascorbic acid, coenzyme F420, adenosine triphosphate, S-adenosyl methionine, 3′-phosphoadenosine-5′-phosphosulfate, coenzyme Q, tetrahydrobiopterin, cytidine triphosphate, nucleotide sugars, glutathione, coenzyme M, coenzyme B, methanofuran, tetrahydromethanopterin, flavin mononucleotide, flavin adenine dinucleotide, pyrroloquinoline quinone, pyridoxal phosphate, biotin, methylcobalamin, thiamine pyrophosphate, heme, molybdopterin, lipoic acid and any derivatives or analogs thereof.

In one embodiment, the process of the invention is carried out in a buffer. Preferably, the buffer has a pH of from about 6 to about 8, more preferably from about 6 to about 7. Preferably, the buffer is a solution of a salt. Preferably, the salt is selected from the group consisting of potassium phosphate, magnesium sulfate, and mixtures thereof. Preferably the buffer is potassium phosphate. Optionally, the buffer comprises a thiol. Preferably, the thiol is DTT. Preferably, the thiol reduces at least one disulfide bond in the enzyme.

In one embodiment, the process of the invention is carried out at a temperature of about 10° C. to about 45° C. The process may be carried out, for example, at room temperature, at a temperature of about 20° C. to about 30° C., or at about 25° C. to about 35° C. Preferably, the process is carried out at a temperature of about 25° C. to about 35° C., such as at a temperature of about 30° C.

In one embodiment, the process of the invention is carried out in the presence of a solvent, such as an organic solvent. Preferably, the organic solvent is water-miscible, such as water-miscible alcohols, water miscible ethers, acetonitrile, tetrahydrofuran, and dimethylsulfoxide. Preferably, the alcohol is a C₁-C₄ alcohol, more preferably methanol or IPA (iso-propyl alcohol). Most preferably, the solvent is dimethoxyethane. With a water-miscible solvent, particularly alcohols and dimethylsulfoxide, preferably the reaction medium is mostly water, which makes the reaction more environmentally friendly.

The process can comprise the following steps: (a) dissolving compound C, and more preferably, compound C(i) in a solvent; and (b) combining the solution from (a) with a buffer containing an enzyme, and optionally a co-factor. Optionally, the solution comprises a co-factor regeneration system. Preferably, the obtained mixture is maintained for a period of time sufficient to obtain (S)-D. Preferably, the reaction is maintained at a temperature of about 10° C. to about 50° C., more preferably about 20° C. to about 40° C., even more preferably at a temperature of about 25° C. to about 35° C., or about 30° C. Preferably, the reaction is maintained for about 24 hours or more, for example about 48 hours or more or about 72 hours or more. More preferably, the reaction is maintained for about 24 hours to about 50 hours. Most preferably, the reaction is maintained for about 24 hours to about 30 hours. The reaction can be stirred.

Optionally, after the reaction is completed, compound (S)-D is isolated by adding an inorganic base solution selected from the group consisting of sodium carbonate, sodium bicarbonate, potassium carbonate, potassium bicarbonate, sodium hydroxide, and potassium hydroxide, preferably sodium bicarbonate. The aqueous solution is then acidified using an inorganic acid, to allow the precipitation of the product. The inorganic acid may be selected from the group consisting of hydrochloric acid, sulfuric acid, nitric acid, hydrobromic acid, and the like. Organic acids like acetic acid or formic acid may also be used. Most preferably, the inorganic acid is hydrochloric acid. The obtained product may be dried under vacuum at a temperature of about 50° C. to about 65° C., preferably about 60° C.

The invention further provides a process for preparing ramelteon, comprising preparing (S)-D with the enzymatic hydrolysis process of the invention, and converting the (S)-D into ramelteon. The (S)-D may be converted into ramelteon by any method known in the art; for example, by the method referred to in WO2008/151170, hereby incorporated by reference.

Preferably, high performance liquid chromatography (HPLC) methods are used to determine the chemical purity of compound (S)-D. The HPLC method may comprise analyzing a sample of compound D by HPLC under the following conditions:

Column: Thermo Hypersil Gold C8, 3.0μ, 150×4.6 mm,

Waters P/N: 25203-154630 or equivalent

Flow: 1.5 ml/min.

Injection Volume: 10 μl

Detector: 220 nm

Column Temperature: 15° C.

Diluent: Eluent A—Buffer, Eluent B—Acetonitrile; 9:1 ratio.

Having described the invention with reference to certain preferred embodiments, other embodiments will become apparent to one skilled in the art from consideration of the specification. The invention is further defined by reference to the following examples. It will be apparent to those skilled in the art that many modifications, both to materials and methods, may be practiced without departing from the scope of the invention.

### EXAMPLES

### Chiral HPLC Method Conditions

Column: Chiral PAK ADH (250×4.6) mm, 5μ

Mobile Phase: n-Heptane:Ethanol (95:5)

Diluent: n-heptane:Ethanol (50:50)

UV: 288 min

Run time: 30 min

Inj. Vol: 10 μL

Flow: 0.8 ml/min

Column oven: 15° C.

Sample Preparation: 500 ppm

### HPLC Method Conditions for Chromatographic Purity:

Column: Xterra RP8, 3.5μ, 150×4.6 mm, Waters, P/N: 186000443 or equivalent.

Flow: 1.5 ml/min

Injection volume: 10 μl

Detector: 217 nm

Column Temperature: 30° C.

Equilibrium time: 10 minutes

Diluent: Acetonitrile

### Example 1

### Synthesis of Intermediate B

A 60% suspension of sodium hydride in mineral oil (22.4 g, 0.560 mol) was added to dry toluene (3000 ml) under N₂ atmosphere at 0-5° C. and stirred for 20 minutes. triethyl phosphonoacetate (168 g, 0.749 mol) was added drop-wise at 15° C. and stirred for 2 hours at 30° C. 4,5-Dibromo-1,2,6,7-tetrahydro-8H-indeno[5,4-b]furan-8-one (100 g, 0.300 mol) was added, and the reaction mixture was heated to 90-100° C. and stirred under N₂ atmosphere for 16 hours, then the reaction mixture was cooled to 30° C., and 1500 ml of water were added. The organic layer was separated and washed with brine solution. The organic layer was distilled off under vacuum at 50° C.

### Example 2

### Synthesis of Intermediate C

Ethyl (2E)-(4,5-dibromo-1,2,6,7-tetrahydro-8H-indeno[5,4-b]furan-8-ylidene) acetate (100 g, 0.301 mol) was dehalogenated and the double bond reduced in methanol (500 ml) in presence of sodium acetate (61.75 g, 0.753 mol) and 10% Pd/C (10.0 g) in a hydrogenator, under a hydrogen atmosphere at a pressure of 4 kg/cm², at 25° C. for 4 hours, and at 55° C. for an additional hour. The reaction mixture was filtered through CELITE HYFLO™ filter aid, and water (100 ml) and sodium hydroxide (24.0 g, 0.60 mol) added. Methanol was removed under vacuum at 50° C., water (1000 ml) was added, and the mixture acidified with aqueous HCl to a pH of 2 at 30° C. The obtained 1,2,6,7-tetrahydro-8H-indeno[5,4-b]furan-8-yl)acetic acid was isolated by filtration, and dried under vacuum at 55° C. Yield: 87.05%. Purity: 98.86%.

### Example 3

### Synthesis of Intermediate E

100 gr of Ethyl (2E)-(4,5-dibromo-1,2,6,7-tetrahydro-8H-indeno[5,4-b]furan-8-ylidene) acetate and Ru(OAc)₂[(R)—BINAP] (1.5 mol) are charged in 2000 ml methanol in an autoclave, and the reaction mixture is pressurized with hydrogen at 3 kg/cm² and stirred for 8 hours. Then, the reaction, the reaction mixture is concentrated under reduced pressure. 500 ml hexane are added at 25° C. and the reaction mixture is stirred for 1 hour. Then, the reaction mixture is cooled to 0° C., and stirred at this temperature for 1 hour. The obtained compound E is filtered out form the reaction mixture.

### Example 4

### Synthesis of Intermediate F

ethyl [(8S)-4,5-dibromo-1,6,7,8-tetrahydro-2H-indeno[5,4-b]furan-8-yl]acetate (100 gr, 0.265 mol), sodium acetate (54 gr, 0.662 mol) and 10% Pd/C (15 gr) in 500 ml of acetic acid are stirred under a hydrogen atmosphere (2-3 kg/cm²) for 4 hours at 30° C. The reaction mixture is filtered through a filter aid (CELITE HYFLO™), and the acetic acid is removed under vacuum at 50-60° C. 500 ml of methanol are added, and the mixture is cooled to 15° C. The obtained ethyl (8S)-1,6,7,8-tetrahydro-2H-indeno[5,4-b]furan-8-ylacetate is isolated by filtration and dried under vacuum.

### Example 5

### Synthesis of Intermediate (S)-D

Ethyl (8S)-1,6,7,8-tetrahydro-2H-indeno[5,4-b]furan-8-ylacetate (100 gr, 0.406 mol) was dissolved in 800 ml methanol, sodium hydroxide was added (24.4 gr, 0.609 mol), and the reaction mixture was stirred for 2-3 hours at 30° C. to effect hydrolysis. The methanol was distilled of under vacuum, water was added, and the mixture was acidified by dropwise addition hydrochloric acid under cooling. The obtained (8S)-1,2,6,7-tetrahydro-8H-indeno[5,4-b]furan-8-yl)acetic acid was isolated by filtration and dried under vacuum. Yield—86% and purity 99.8%.

### Example 6

A solution of (8S)-1,2,6,7-tetrahydro-8H-indeno[5,4-b]furan-8-yl)acetic acid

(35.0 gr, 0.1605 mol) in 350 ml of dichloromethane was cooled to between −10° C. Triethylamine (19.25 gr, 0.1905 mol) was added dropwise to the reaction mixture, and the obtained reaction mixture was maintained at 0° C. The reaction mixture was cooled to −10° C. and ethyl chloroformate (19.95 gr, 0.1838 mol) was added dropwise to the reaction with cooling, which was then stirred for 2 hours at 0-5° C. Then, the reaction mixture was quenched in 550 ml of 2-5% ammonia solution in dichloromethane at 0-5° C. The reaction mixture was stirred for an hour 1 at 0-5° C. The solvent was distilled out under vacuum at 40-45° C. and 245 ml of water and sodium bicarbonate (8.75 gr) were added. 2-[(8S)-1,6,7,8-tetrahydro-2H-indeno[5,4-b]furan-8-yl]acetamide was isolated by filtration and washed with water. Yield: 94%. Purity: 99.79%.

### Example 7

### Process-I (Hydrochloride Salt)

Sodium borohydride (74.2 g, 1.96 mol) was added to a stirred solution of BF₃ etherate (247.8 ml) in THF (1800 ml) at −10° C. The reaction mixture was stirred for 3 hours at 0-5° C., and then 2-[(8S)-1,6,7,8-tetrahydro-2H-indeno[5,4-b]furan-8-yl]acetamide (100 g, 0.460 mol) was added. The reaction was stirred at 55° C. for 15 hours. The reaction mixture was quenched in 3600 ml water and 200 ml concentrated hydrochloric acid, and the THF was removed under vacuum at 50° C. The reaction mixture was diluted with toluene, and basified with NaOH to pH 11. The organic layer was separated, washed with brine and sodium carbonate solution, and concentrated. HCl gas was passed into the resulting solution, and the precipitated salt was filtered and dried under vacuum at 50-55° C. Yield: 82.79%. Purity: 99.41%.

### Process-II (Oxalate Salt)

Sodium borohydride (74.2 g, 1.96 mol) was added to a stirred solution of BF₃ etherate (247.8 ml) in THF (1800 ml) at −10° C. The reaction mixture was stirred for 3 hours at 0-5° C., and then 2-[(8S)-1,6,7,8-tetrahydro-2H-indeno[5,4-b]furan-8-yl]acetamide (100 g, 0.461 mol) was added. The reaction was stirred at 40-45° C. for 15 hours. The reaction mixture was quenched in 3600 ml water and 200 ml concentrated hydrochloric acid, and THF was removed under vacuum at 50° C. The reaction mixture was diluted with toluene, and basified with NaOH to pH 11. The organic layer was separated, washed with brine and sodium carbonate solution, and concentrated. A solution of oxalic acid in methanol was added, the mixture was cooled to 0-5° C., and the precipitated solid was isolated by filtration, washed, and dried under vacuum at 55° C. Yield: 85%. Purity: 98%.

### Example 8

The oxalate salt of 2-[(8S)-1,6,7,8-tetrahydro-2H-indeno[5,4-b]furan-8-yl]ethanamine (100.0 g, 0.3759 mol) was stirred into a solution of sodium carbonate (1120 g, 1.127 mol) in water (600 ml) and dichloromethane (1000 ml) at 30° C. The reaction mixture was cooled to −5 to 10° C., propionyl chloride (51.02 g, 0.5638 mol) in dichloromethane was added dropwise, and the mixture was stirred for 1 hour. The organic layer was separated and washed with sodium bicarbonate and 10% brine solution. The organic layer was evaporated, and the isolated compound. The compound was purified by crystallization from 200 ml ethanol. Yield: 80%. Purity: 99.5%.

### Example 9

The oxalate salt of 2-[(8S)-1,6,7,8-tetrahydro-2H-indeno[5,4-b]furan-8-yl]ethanamine (100.0 g, 0.4936 mol) was stirred into a solution of sodium carbonate (130.0 g, 1. 231 mol) in water (600 ml) and dichloromethane (1000 ml) at 30° C. The reaction mixture was cooled to −5 to 10° C., propionyl chloride (51.02 g, 0.5638 mol) in dichloromethane was added dropwise, and the mixture was stirred for 1 hour. The organic layer was separated and washed with sodium bicarbonate and 10% brine solution. The organic layer was evaporated, and the isolated compound. The compound was purified by crystallization from 200 ml ethanol. Yield: 80%. Purity: 99.5%.

### Example 10

The hydrochloride salt of 2-[(8S)-1,6,7,8-tetrahydro-2H-indeno[5,4-b]furan-8-yl]ethanamine (100.0 g, 0.4936 mol) was stirred into a solution of sodium carbonate (130.0 g, 1. 231 mol) in water (600 ml) and dichloromethane (1000 ml) at 30° C. The reaction mixture was cooled to −5 to 10° C., propionyl chloride (51.02 g, 0.5638 mol) in dichloromethane was added dropwise, and the mixture was stirred for 1 hour. The organic layer was separated and washed with sodium bicarbonate and 10% brine solution. The organic layer was evaporated, and the isolated compound. The compound was purified by crystallization from 200 ml ethanol. Yield: 80%. Purity: 99.5%.

### Example 11

### Preparation of Compound H

An azeotropic distillation was perform by adding 4,5-dibromo-1,2,6,7-tetrahydro-8H-indeno[5,4-b]furan-8-one (100.0 g 0.3012 mol) in toluene (2000 ml) to remove traces of water. The reaction mixture was cooled to 0-5° C. and diethyl cyanomethyl phosphonate (123.0 g, 0.6944 mol) was added. The freshly prepared 28% sodium methoxide solution (165 ml) was added dropwise into reaction mixture with stirring and maintaining the temperature 0-5° C. The reaction mixture was poured into water with stirring and separated out the organic layer. The organic layer was dried and distilled out to isolate the obtained (2E)-(4,5-dibromo-1,2,6,7-tetrahydro-8H-indeno[5,4-b]furan-8-ylidene)acetonitrile. Yield 85%. Purity 95%.

### Example 12

### Preparation of Compound H

An azeotropic distillation was perform by adding 4,5-dibromo-1,2,6,7-tetrahydro-8H-indeno[5,4-b]furan-8-one (100.0 g 0.3012 mol) in toluene (2000 ml) to remove traces of water. The reaction mixture was cooled to 0-5° C. and diethyl cyanomethyl phosphonate (64 g, 0.3614 mol) was added. The freshly prepared 28% sodium methoxide solution (165 ml) was added dropwise into reaction mixture with stirring and maintaining the temperature 0-5° C. The reaction mixture was poured into water with stirring and separated out the organic layer. The organic layer was dried and distilled out to isolate the obtained (2E)-(4,5-dibromo-1,2,6,7-tetrahydro-8H-indeno[5,4-b]furan-8-ylidene)acetonitrile. Yield 85%. Purity 95%.

### Example 13

### Preparation of Compound I

A 30% hydrogen peroxide solution (500 ml) was added dropwise to a solution of (2E)-(4,5-dibromo-1,2,6,7-tetrahydro-8H-indeno[5,4-b]furan-8-ylidene)acetonitrile (100.0 g, 0.2816 mol) and potassium hydroxide (300 g) in dimethylsulfoxide (1000 ml) and water (1200 ml). The mixture was then stirred at room temperature for 4 hours. The reaction mixture was poured in water and extracted with ethyl acetate. The extract was dried and concentrated under reduced pressure to give solids, which was crystallized using ethyl acetate to give 2-(4,5-dibromo-1,6-dihydro-2H-indeno[5,4-b]furan-8-yl)acetamide. Yield 40%. Purity: 95%.

### Example 14

### Preparation of Compound K

A solution of (2E)-(4,5-dibromo-1,2,6,7-tetrahydro-8H-indeno[5,4-b]furan-8-ylidene)acetonitrile (100.0 g, 0.2816 mol) and Ru(OAc)₂-[(R)—BINAP] (19.63 g, 0.0234 mol) in ethanol (700 ml) is charged into a auto clave and reaction mass is flux twice with hydrogen. The 10 MPa pressure of hydrogen is applied and reaction mass is stirred at 50° C. The reaction mixture is concentrated under reduced pressure to isolated [(8S)-4,5-dibromo-1,6,7,8-tetrahydro-2H-indeno[5,4-b]furan-8-yl]acetonitrile.

### Example 15

### Preparation of Compound J

A solution of 2-(4,5-dibromo-1,6-dihydro-2H-indeno[5,4-b]furan-8-yl)acetamide (100 g, 0.281 mole) and Ru(OAc)₂-[(R)—BINAP] (19.63 g, 0.0234 mol) in ethanol (700 ml) was charged into a auto clave and reaction mass was flux twice with hydrogen. The 10 MPa pressure of hydrogen was applied and reaction mass was stirred at 50° C. The reaction mixture was concentrated under reduced pressure to isolated 2-[(8S)-4,5-dibromo-1,6,7,8-tetrahydro-2H-indeno[5,4-b]furan-8-yl]acetamide. Yield: 85%. Purity: 95.5%. Chiral purity is 90%

### Example 16

### Preparation of Compound L

### Process-I (Hydrochloride Salt):

Sodium borohydride (74.2 g, 1.9631 mol) was added into stirred solution of BF₃ etherate (247.8 ml) in THF (1800.0 ml) at −10° C. The reaction mixtures was stirred for 2 hours at 5° C. Then 2-[(8S)-4,5-dibromo-1,6,7,8-tetrahydro-2H-indeno[5,4-b]furan-8-yl]acetamide (100 g, 0.266 mol) was added into it and reaction was stirred at 45-55° C. for 7 hours. The reaction mixture was quenched with 3600 ml water and 200 ml acetic acid. The THF was distilled out under vacuum at 40-50° C. The reaction mixture was diluted with toluene and was basified with liquid ammonia to obtain a pH of 10. The organic layer was separated and washed it with brine and sodium carbonate solution. Concentrate the reaction mixture and passed the HCl gas. The precipitate (2-[(8S)-4,5-dibromo-1,6,7,8-tetrahydro-2H-indeno[5,4-b]furan-8-yl]ethanamine chloride) was filtered and dried under vacuum at 50-55° C. Yield 85%. Purity 99%.

### Example 17

### Preparation of Compound L

### Process-I (Hydrochloride Salt):

Sodium borohydride (34.6 g, 0.9230 mol) was added into stirred solution of BF₃ etherate (144.0 ml) in THF (1800.0 ml) at −10° C. The reaction mixtures was stirred for 2 hours at 5° C. Then 2-[(8S)-4,5-dibromo-1,6,7,8-tetrahydro-2H-indeno[5,4-b]furan-8-yl]acetamide (100 g, 0.266 mol) was added into it and reaction was stirred at 45-55° C. for 7 hours. The reaction mixture was quenched with 3600 ml water and 200 ml acetic acid. The THF was distilled out under vacuum at 40-50° C. The reaction mixture was diluted with toluene and was basified with liquid ammonia to obtain a pH of 10. The organic layer was separated and washed it with brine and sodium carbonate solution. Concentrate the reaction mixture and passed the HCl gas. The precipitate (2-[(8S)-4,5-dibromo-1,6,7,8-tetrahydro-2H-indeno[5,4-b]furan-8-yl]ethanamine chloride) was filtered and dried under vacuum at 50-55° C. Yield 85%. Purity 99%.

### Process-II (Oxalate Salt)

Sodium borohydride (74.2 g, 1.963 μmol) was added into stirred solution of BF3 etherate (247.8 ml) in THF (1800.0 ml) at −10° C. The reaction mixtures was stirred for 2 hours at 5° C. Then 2-[(8S)-4,5-dibromo-1,6,7,8-tetrahydro-2H-indeno[5,4-b]furan-8-yl]acetamide (100 g, 0.266 mol) was added into it and reaction was stirred at 45° C. for 7 hours. The reaction mixture was quenched with 3600 ml water and 200 ml concentrated hydrochloric acid. The tetrahydrofuran was distilled out under vacuum at 45° C. The reaction mixture was diluted with toluene and basify it with NaOH up to 10 pH. The organic layer was separated and washed it with brine and sodium carbonate solution. Concentrate the reaction mixture and added oxalic acid solution in methanol. The 2-[(8S)-4,5-dibromo-1,6,7,8-tetrahydro-2H-indeno[5,4-b]furan-8-yl]ethanamine oxalate precipitated and was cooled to 5° C. and filtered, wash and dried under vacuum at 50-55° C. Yield 85%. Purity 99%.

### Process-II (Oxalate Salt)

Sodium borohydride (34.6 g, 0.9230 mol) was added into stirred solution of BF3 etherate (144 ml) in THF (1800.0 ml) at −10° C. The reaction mixtures was stirred for 2 hours at 5° C. Then 2-[(8S)-4,5-dibromo-1,6,7,8-tetrahydro-2H-indeno[5,4-b]furan-8-yl]acetamide (100 g, 0.266 mol) was added into it and reaction was stirred at 45° C. for 7 hours. The reaction mixture was quenched with 3600 ml water and 200 ml concentrated hydrochloric acid. The tetrahydrofuran was distilled out under vacuum at 45° C. The reaction mixture was diluted with toluene and basify it with NaOH up to 10 pH. The organic layer was separated and washed it with brine and sodium carbonate solution. Concentrate the reaction mixture and added oxalic acid solution in methanol. The 2-[(8S)-4,5-dibromo-1,6,7,8-tetrahydro-2H-indeno[5,4-b]furan-8-yl]ethanamine oxalate precipitated and was cooled to 5° C. and filtered, wash and dried under vacuum at 50-55° C. Yield 85%. Purity 99%.

### Example 18

### Preparation of Compound K

A mixture of 2-[(8S)-4,5-dibromo-1,6,7,8-tetrahydro-2H-indeno[5,4-b]furan-8-yl]acetamide (10 gr, 0.027 mol) in 100 ml of toluene was heated at 85° C. in presence of POCl₃ (5.3 gr, 0.035 mol) for 5 hours. The reaction mixture was poured into 250 ml of ice cold water with stirring. The mixture was extracted with ethyl acetate. The organic layer was wash with 5% sodium bicarbonate solution and then water. Organic layer was decolorized by charcoal then dried and distilled out under reduced pressure to get [(8S)-4,5-dibromo-1,6,7,8-tetrahydro-2H-indeno[5,4-b]furan-8-yl]acetonitrile. Yield. 75%. Purity: 98%.

### Example 19

### Preparation of Compound L

To a mixed suspension of [(8S)-4,5-dibromo-1,6,7,8-tetrahydro-2H-indeno[5,4-b]furan-8-yl]acetonitrile (100 gr) and 200 ml of acetonitrile in 100 ml of toluene and 100 ml of methanol were added Raney Cobalt (30 gm 50% wet) and 100 ml of 14.4% aqueous solution of potassium hydroxide and stirred for 10 hours at 45° C. under hydrogen pressure 5 kg/cm². The reaction solution was filtered off and the methanol was distilled out. The mixture was washed with water. The separated organic layer was treated with isopropanol and HCl to precipitate the hydrochloric salt of 2-[(8S)-4,5-dibromo-1,6,7,8-tetrahydro-2H-indeno[5,4-b]furan-8-yl]ethanamine. Yield 82.5%. Purity: 97.8%.

### Example 20

### Preparation of Compound RML-XXII

Pd/C (15.0 g) were added to a mixed suspension of [(8S)-4,5-dibromo-1,6,7,8-tetrahydro-2H-indeno[5,4-b]furan-8-yl]ethanamine (100 g, 0.2522 mol), 500 ml methanol, and sodium acetate (51.7 g, 0.6305 mol). The reaction mixture was stirred for 5 hours at 30° C. under hydrogen pressure 5 kg/cm². The reaction solution was filtered off and the methanol was distilled out. Isopropanol and HCl were added to precipitate the hydrochloric salt of 2-[(8S)-1,6,7,8-tetrahydro-2H-indeno[5,4-b]furan-8-yl]ethanamine. Yield 82.5%. Purity: 99.5%.

### Example 21

### Preparation of Compound M

A mixture of (2E)-(4,5-dibromo-1,2,6,7-tetrahydro-8H-indeno[5,4-b]furan-8-ylidene)acetonitrile (25 g, 0.07 mol) and Raney Co (18.5 g) with 187.5 ml of toluene and 100 ml of methanol was stirred at 55° C. and 5 kg/cm² of hydrogen gas pressure. The reaction mixture was filtered through hyflow and distilled out methanol and toluene completely under reduce pressure added toluene and isopropanol and HCl to form the hydrochloric salt of compound M. The mixture was stirred at 30° C. for about 30 minutes and the product was filtered out and washed with toluene. Yield: 75%. Purity: 90%.

### Example 22

### Preparation of Compound N

(2E)-2-(4,5-dibromo-1,2,6,7-tetrahydro-8H-indeno[5,4-b]furan-8-ylidene)ethanamine hydrochloride salt (10.0 g, 0.0252 mol) was added to a mixture of Sodium carbonate (5.34 g, 0.0504 mol) in water (50 ml) and toluene stirred and separate organic layer, organic layer dehalogenated and reduce double bond in methanol (50 ml) in presence of sodium acetate (5.16 g, 0.063 mol) and 10% Pd/C (1.5 g) in hydrogenator in pressure with 4.5 kg/cm² and 50° C. The reaction mixture was filtered through hyflow bed and the solvents were distilled off completely. Toluene and IPA HCl were added until a pH of 2 was reached. The mixture was stirred at 30° C. for 2 hours. The product was filtered and washed with toluene. Yield: 87%. Purity: 98.5%.

The hydrochloric salt of (2E)-2-(4,5-dibromo-1,2,6,7-tetrahydro-8H-indeno[5,4-b]furan-8-ylidene)ethanamine was stirred with a sodium carbonate solution (5.34 g, 0.0504 mol) and toluene. The organic layer was separated from the aqueous layer to obtain (2E)-2-(4,5-dibromo-1,2,6,7-tetrahydro-8H-indeno[5,4-b]furan-8-ylidene)ethanamine free base.

Chiral reduction of (2E)-2-(4,5-dibromo-1,2,6,7-tetrahydro-8H-indeno[5,4-b]furan-8-ylidene)ethanamine free amine (10 g) with chiral Ruthenium BINAP (S) (0.5 g) in methanol/toluene (10/15) at 80° C. under 8.5 kg/cm² of hydrogen gas pressure for 4 hours. 10% Pd/C (1.5 g) and sodium acetate (0.06963 mol) were added to the reaction mixture. The mixture was heat to 55° C. under hydrogen pressure of 5 kg/cm² for 3 hours. The solvents were evaporated under reduced pressure at 50° C. to obtain 2-[(8S)-1,6,7,8-tetrahydro-2H-indeno[5,4-b]furan-8-yl]ethanamine. Yield: 75.8%. Chiral Purity 98.5% (S-isomer).

### Example 23

### Preparation of Ramelteon

2-[(8S)-1,6,7,8-tetrahydro-2H-indeno[5,4-b]furan-8-yl]ethanamine hydrochloride salt (100.0 g, 0.4175 mol) was stirred in sodium carbonate (110.6 g, 1.043 mol) solution in water (600.0 ml) and dichloromethane (1000.0 ml) at 25° C. The reaction mixture was cooled up to 0° C. for an hour. Propionyl chloride (40.5 g, 0.4383 mol) in dichloromethane was added dropwise into reaction mixture and stirred it for 1 hour. The organic layer was separated and washed it with sodium bicarbonate and 10% brine solution. The organic layer was distilled out and the obtained ramelteon was isolated by filtration. The isolated compound was purified from ethanol. Yield: 80%. Purity: 99.5%.

### Example 24

A mixture of 2-[(8S)-1,6,7,8-tetrahydro-2H-indeno[5,4-b]furan-8-yl]acetamide (25 gr, 0.11 mol) in 125 ml toluene was heated at 80-85° C. in presence of POCl₃ (16.8 gr, 0.15 mol) for 5 hours. The reaction mixture was poured into 500 ml of ice cold water with stirring. The mixture was extracted with ethyl acetate. The Organic layer was washed with 5% sodium bicarbonate solution and then water. The organic layer was decolorized by charcoal then dried and distilled out under reduced pressure to get (8S)-1,6,7,8-tetrahydro-2H-indeno[5,4-b]furan-8-ylacetonitrile. Yield: 70%. Purity: 98%.

### Example 25

(S)-(4,5-dibromo-1,2,6,7-tetrahydro-8H-indeno[5,4-b]furan-8-yl)acetic acid was synthesized from ethyl (2E)-(4,5-dibromo-1,2,6,7-tetrahydro-8H-indeno[5,4-b]furan-8-yl)acetate (10.0 g, 0.04 mol) in a binary mixture of buffer A (50.0 ml) (pH=7.0) and dimethoxy ethane (50.0 ml) in the presence of NZL-102-LYO (4.0 g) The reaction mixture was stirred at 30° C. for 48 hours. (S)-(4,5-dibromo-1,2,6,7-tetrahydro-8H-indeno[5,4-b]furan-8-yl)acetic acid was isolated by adding 50 ml of 10% sodium bicarbonate solution with stirring. The sodium bicarbonate aqueous layer was acidified with 45 ml of 10% HCl up to a pH of 2.0, and the precipitate was isolated and dried under vacuum at 60° C. Yield: 45%.

### Example 26

(S)-(1,2,6,7-tetrahydro-8H-indeno[5,4-b]furan-8-yl)acetic acid was synthesized from ethyl (2E)-(1,2,6,7-tetrahydro-8H-indeno[5,4-b]furan-8-yl)acetate (10.0 g, 0.04 mol) in binary mixture of buffer A (50.0 ml) and dimethoxyethane (50.0 ml) in the presence NZL-102-LYO (4.0 gm). The reaction mixture was stirred at 30° C. for 48 hours. 10% sodium bicarbonate was added to reaction mixture until the pH=8 of solution attained. The sodium bicarbonate aqueous layer was acidified with HCl until the reaction mixture achieves pH=2 and the precipitate was isolated and dried under vacuum at 60° C. Yield 80% of the (S) isomer.

### HPLC Analysis:

(a) Chemical Purity: 95%

(b) Enantiomeric Purity:

TABLE 1

| | | | | Enantiomeric | Enantiomeric |
|---|---|---|---|---|---|
| | | | | purity | purity |
| | | | | (of compound | (of compound |
| | | | | D wherein | D wherein |
| S.N. | Buffer | Solvent | Enzyme | x = Br) | x = H) |
| 01 | Buffer A | Dimethoxyethane | NZL-102-LYO | 85% | 92% |
| | (potassium | | | | |
| | phosphate) | | | | |
| 02 | Buffer A | Dimethoxyethane | NZL-103-LYO | 85% | 90% |
| | (potassium | | | | |
| | phosphate) | | | | |
| 03 | Buffer A | Dimethoxyethane | NZL-107-LYO | 80% | 85% |
| | (potassium | | | | |
| | phosphate) | | | | |

TABLE 2
Enzyme Lot Nos., and years of production:

| Product Number | Lot Number | Production Year |
|---|---|---|
| NZL-102-LYO | Lot#021607IM | 2007 |
| NZL-103-LYO | Lot#613107IM | 2007 |
| NZL-107-LYO | Lot#713107IM | 2007 |

1. A compound B, having one or more of the following isomer structure: 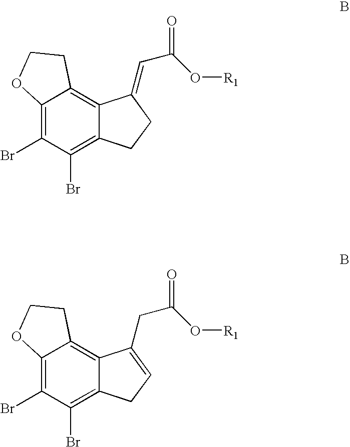
wherein, R1 is selected from the group consisting of C₁-C₆ straight or branched alkyls, C₆-C₁₀ aryls, and alkylaryl, wherein the alkyl contains 1-4 carbons, and the aryl contains 6-12 carbons.

2. A process for preparing the compound of claim 1, comprising contacting compound of the formula A: 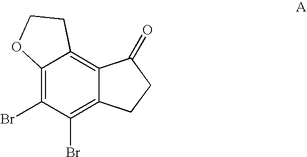
with a trialkylphosphonoacetate.

3. The process of claim 2, wherein the alkyl group of the trialkylphosphonoacetate can be the same or different and is selected from the group consisting of C₁-C₆ straight or branched alkyls, C₆-C₁₀ aryls, and arylalkyls wherein the alkyl contains 1-4 carbons, and the aryl contains 6-12 carbons.

4. The process of claim 2, wherein the reaction is carried out in the presence of a base selected from the group consisting of alkali metal hydroxide, metal amides, metal alkoxides, alkyllithiums, amine bases, and alkali metal hydrides.

5. The process of claim 2, wherein the reaction is carried out in the presence of an organic solvent selected from the group consisting of one or more of C₆-C₁₀ substituted aromatic hydrocarbons, and C₁-C₅ halogenated hydrocarbons.

6. The process of claim 2, further comprising isolating the compound of claim 1.

7. A process of preparing Ramelton comprising converting the compound B of claim 2 to ramelteon.

8. A process for preparing compound C, of the following formula: 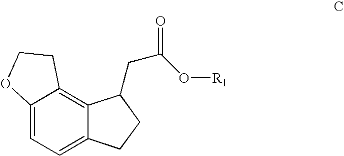
comprising reducing the double bond in the compound of formula B, and dehalogenation of the bromo groups, wherein either reaction may precede the other.

9. The process of claim 8, wherein the reduction of the double bond is carried out by catalytic reduction with hydrogen.

10. The process of claim 8, wherein the reaction is conducted in the presence of Pd—C, Raney-Ni, Zn/HCl or Fe/HCl

11. The process of claim 8, wherein the reaction is conducted in a solvent selected from the group comprising of one or more of C₁ to C₆ halogenated hydrocarbons, C₆ to C₁₄ aromatic hydrocarbons, C₁ to C₅ alcohols, C₂ to C₇ esters, C₄ to C₇ ethers, C₁ to C₅ carboxylic acids, C₅ to C₈ cyclic ethers, water, and suitable mixtures thereof.

12. The process of claims 11, wherein the solvent is selected from the group consisting of one or more of methanol, isopropyl alcohol, dichloromethane, toluene, ethyl acetate, and diethyl ether.

13. A process of preparing Ramelton comprising converting the compound C of claim 8 to ramelteon.

14. A process for preparing compound D, of the following formula: 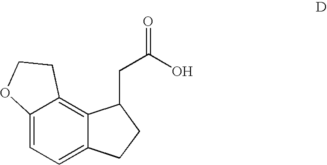
comprising acidic or basic hydrolysis of compound C.

15. The process of claim 14, further comprising converting the compound D to ramelteon.

16. A compound E having the structure: 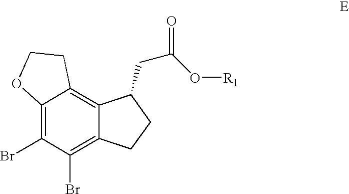
wherein, R₁ is selected from the group consisting of C₁-C₆ straight or branched alkyls, C₆-C₁₀ aryls, and alkylaryl, wherein the alkyl contains 1-4 carbons, and the aryl contains 6-12 carbons.

17. A process for preparing the compound of claim 16, comprising stereoselectively reducing double bond of compound B.

18. The process of claim 17, wherein the reduction is carried out in the presence of a catalyst selected from the group consisting of (+)-Ru(OAc)₂[(R)—BINAP], (+)-Ru(Cl₂)benzene-[(R)—BINAP], and (+)-Ru(Cl₂)p-cymene-[(R)—BINAP], with (+)-Ru(OAc)₂[(R)—BINAP].

19. The process of claim 18, wherein the catalyst is (+)-Ru(OAc)₂[(R)—BINAP].

20. The process of claim 18, wherein the reaction is conducted in a solvent selected from the group consisting of one or more of C₁ to C₆ halogenated hydrocarbons, C₆ to C₁₄ aromatic hydrocarbons, C₁ to C₅ alcohols, C₂ to C₇ esters, C₄ to C₇ ethers, C₁ to C₅ carboxylic acids, water, or suitable mixtures of these solvents.

21-88. (canceled)
